(19) United States
(12) Patent Application Publication
(10) Pub. No.: US 2024/0339850 A1
(43) Pub. Date: Oct. 10, 2024

(54) NEAR-FIELD-COMMUNICATION-BASED POWER CONTROL FOR AN ANALYTE SENSOR SYSTEM

(71) Applicant: Dexcom, Inc., San Diego, CA (US)

(72) Inventor: Didier SAGAN, San Diego, CA (US)

(21) Appl. No.: 18/628,303

(22) Filed: Apr. 5, 2024

Related U.S. Application Data
(60) Provisional application No. 63/494,980, filed on Apr. 7, 2023.

(51) Int. Cl. H02J 7/00; A61B 5/00; A61B 5/145; H02J 50/00

(52) U.S. Cl. CPC H02J 7/0063; A61B 5/002; A61B 5/14546; A61B 5/743; H02J 50/001; A61B 5/14532; A61B 2562/12

(57) ABSTRACT

The present disclosure provide techniques for near field communication (NFC)-based power control for an analyte sensor system. The analyte sensor system may include a power control sensor configured to detect whether a signal is applied to the power control sensor and, based on the detection, operate a power switch to control current flow from a battery of the analyte sensor system. The power control sensor may be configured to receive a first control signal from an analog front end (AFE) component of the analyte sensor system instructing the power control sensor to operate the power switch. The AFE component may be configured to receive a second set of control signals from an NFC reader device or one or more other electrical components of the analyte sensor system instructing the AFE component to output the first set of control signals for instructing the power control sensor to operate the power switch.

### CROSS-REFERENCE TO RELATED APPLICATIONS

This application claims priority to and benefit of U.S. Provisional Patent Application No. 63/494,980, filed Apr. 7, 2023, which is assigned to the assignee hereof and hereby expressly incorporated herein in its entirety as if fully set forth below and for all applicable purposes.

### TECHNICAL FIELD

The present disclosure relates generally to an electronic device, such as an analyte sensor system for monitoring analyte values of a user. More particularly, the present disclosure is directed to power control techniques for the analyte sensor system.

### BACKGROUND

Diabetes is a metabolic condition relating to the production or use of insulin by the body. Insulin is a hormone that allows the body to use glucose for energy, or store glucose as fat. When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher-than-normal blood sugar level is called “hyperglycemia.” Chronic hyperglycemia can lead to several of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce adequate insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

### SUMMARY

One aspect of the present disclosure provides an analyte sensor system, comprising: a power switch; a power control sensor; an analog front end (AFE) component; and one or more other electrical components, wherein: the power control sensor is configured to detect whether a signal is applied to the power control sensor and, based on the detection, operate the power switch to control current flow from a battery of the analyte sensor system to at least a transcutaneous analyte sensor of the analyte sensor system, the AFE component, and the one or more other electrical components; the power control sensor is configured to receive a first set of control signals from the AFE component for instructing the power control sensor to operate the power switch; the AFE is configured to receive a second set of control signals from at least one of a near field communication (NFC) reader device or the one or more other electrical components instructing the AFE to output the first set of control signals for instructing the power control sensor to operate the power switch.

Another aspect of the present disclosure provides a method for operating a power switch at an analyte sensor system. The method includes detecting, by a power control sensor of the analyte sensor system, whether a signal is applied to the power control sensor; receiving, by an analog front end (AFE) component of the analyte sensor system from at least one of a near field communication (NFC) reader device or one or more other electrical components of the analyte sensor system, a second set of control signals for instructing the power control sensor to operate a power switch to control current flow from a battery of the analyte sensor system to at least the transcutaneous analyte sensor, the AFE component, and the one or more other electrical components; outputting, by the AFE component based on the second set of control signals, a first set of control signals for instructing the power control sensor to operate the power switch; receiving, by the power control sensor, the first set of control signals from the AFE component; and operating, by the power control sensor based on the first set of control signals and the detection of whether the signal is applied to the power control sensor, the power switch to control the current flow from the battery of the analyte sensor system to at least the transcutaneous analyte sensor, the AFE component, and the one or more other electrical components.

Another aspect of the present disclosure provides an analyte monitoring system, comprising: an analyte sensor system configured to perform analyte measurements associated with a user of the analyte sensor system; and a display device configured to receive the analyte measurements from the analyte sensor system, wherein: the analyte sensor system includes: a power switch; a power control sensor; an analog front end (AFE) component; and one or more other electrical components; the power control sensor is configured to detect whether a signal is applied to the power control sensor and, based on the detection, operate the power switch to control current flow from a battery of the analyte sensor system to at least a transcutaneous analyte sensor of the analyte sensor system, the AFE component, and the one or more other electrical components; the power control sensor is configured to receive a first set of control signals from the AFE component for instructing the power control sensor to operate the power switch; the AFE is configured to receive a second set of control signals from at least one of a near field communication (NFC) reader device or the one or more other electrical components instructing the AFE to output the first set of control signals for instructing the power control sensor to operate the power switch.

Another aspect of the present disclosure provides an analyte sensor system, comprising: a transcutaneous analyte sensor; a battery; a power switch; a power control sensor; an analog front end (AFE) component; and one or more other electrical components, wherein: the transcutaneous analyte sensor is configured to measure one or more analyte levels of a user of the analyte sensor system; the battery is configured to supply current to at least the transcutaneous analyte sensor, the AFE component, and the one or more other electrical components; the power switch is configured provide a path for current flow from the battery to at least the transcutaneous analyte sensor, the AFE component, and the one or more other electrical components; the power control sensor is configured to detect whether a signal is applied to the power control sensor and, based on the detection, operate the power switch to control the current flow from the battery to at least the transcutaneous analyte sensor, the AFE component, and the one or more other electrical components; the power control sensor includes a set of control pins configured to receive a first set of control signals from the AFE component for instructing the power control sensor to operate the power switch; and the AFE is configured to receive a second set of control signals from at least one of a near field communication (NFC) reader device or the one or more other electrical components instructing the AFE to output the first set of control signals for instructing the power control sensor to operate the power switch.

### BRIEF DESCRIPTION OF THE DRAWINGS

Further aspects of the present disclosure will be more readily appreciated upon review of the detailed description of the various disclosed embodiments, described below, when taken in conjunction with the accompanying figures.

FIG. 1 illustrates aspects of an example system that may be used in connection with some embodiments.

FIG. 2 illustrates aspects of an example system that may be used in connection with some embodiments.

FIG. 3A is an example analyte sensor system, in accordance with some embodiments.

FIG. 3B is an example analyte sensor system, in accordance with some embodiments.

FIG. 4 illustrates aspects of an example analyte sensor system, in accordance with some embodiments.

FIG. 5 illustrates aspects of an example analyte sensor system, in accordance with some embodiments.

FIG. 6 illustrates a simplified block diagram of an example analyte sensor system and a power activation module.

FIG. 7 illustrates another simplified block diagram of an example analyte sensor system and a power activation module.

FIG. 8 illustrates a simplified block diagram of an example analyte sensor system having an analog front end (AFE) component configured to operate a power switch in a power control senor using one control pin on the power control sensor.

FIGS. 9A and 9B include an example logic state diagram associated with a power switch of the TMR sensor of an analyte sensor system.

FIG. 10 illustrates a simplified block diagram of an example analyte sensor system having an AFE component configured to operate a power switch in a power control senor using two control pins on the power control sensor.

FIGS. 11A and 11B include an example logic state diagram associated with a power switch of the TMR sensor of an analyte sensor system.

FIG. 12 depicts a method for operating a power switch of an analyte sensor system.

The figures, described in greater detail in the description and examples below, are provided for purposes of illustration only, and merely depict typical or example embodiments of the disclosure. The figures are not intended to be exhaustive or to limit the disclosure to the precise form disclosed. It should also be understood that the disclosure may be practiced with modification or alteration, and that the disclosure may be limited only by the claims and the equivalents thereof.

### DETAILED DESCRIPTION

Aspects of the present disclosure provide systems, methods, and devices for reducing power consumption or dissipation associated with an analyte sensor system.

The details of some example embodiments of the systems, methods, and devices of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, features, and advantages be included within this description (whether explicitly or by reference), be within the scope of the present disclosure, and be protected by one or more of the accompanying claims.

### System Overview and Example Configurations

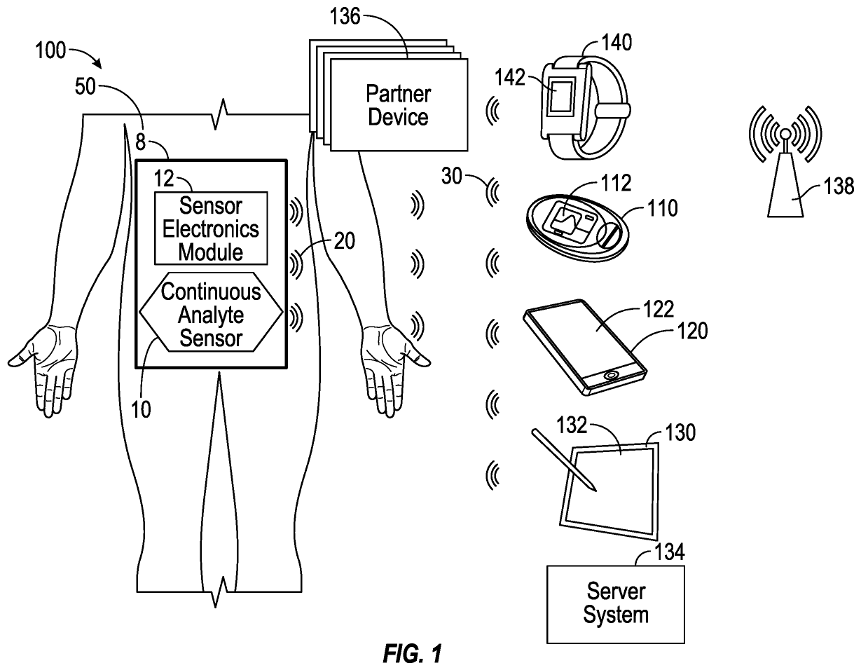

FIG. 1 depicts a system 100 that may be used in connection with embodiments of the present disclosure that involve gathering, monitoring, and/or providing information regarding analyte values present in a user's body, including for example the user's blood glucose values, other analytes, multiple multiplexed or simultaneous measured analytes, or the like. System 100 depicts aspects of analyte sensor system 8 that may be communicatively coupled to display devices 110, 120, 130, and 140, partner devices 136, and/or server system 134.

Analyte sensor system 8 in the illustrated embodiment includes analyte sensor electronics module 12 and analyte sensor 10 associated with analyte sensor electronics module 12. Analyte sensor electronics module 12 may be electrically and mechanically coupled to analyte sensor 10 before analyte sensor 10 is implanted in a user or host. Accordingly, analyte sensor 10 may not require a user to couple analyte sensor electronics module 12 to analyte sensor 10. For example, analyte sensor electronics module 12 may be physically/mechanically and electrically coupled to analyte sensor 10 during manufacturing, and this physical/mechanical and electrical connection may be maintained during shipping, storage, insertion, use, and removal of analyte sensor system 8. As such, the electro-mechanically connected components (e.g., analyte sensor 10 and analyte sensor electronics module 12) of analyte sensor system 8 may be referred to as a “pre-connected” system. Analyte sensor electronics module 12 may be in wireless communication (e.g., directly or indirectly) with one or more of display devices 110, 120, 130, and 140. In addition, or alternatively to display devices 110, 120, 130, and 140, analyte sensor electronics module 12 may be in wireless communication (e.g., directly or indirectly) with partner devices 136 and/or server system 134. Likewise, in some examples, display devices 110-140 may additionally or alternatively be in wireless communication (e.g., directly or indirectly) with partner devices 136 and/or server system 134. Various couplings shown in FIG. 1 can be facilitated with wireless access point (WAP) 138, as also mentioned below.

In certain embodiments, analyte sensor electronics module 12 includes electronic circuitry associated with measuring and processing analyte sensor data or information, including prospective algorithms associated with processing and/or calibration of the analyte sensor data/information. Analyte sensor electronics module 12 can be physically/mechanically connected to analyte sensor 10 and can be integral with (non-releasably attached to) or releasably attachable to analyte sensor 10. Analyte sensor electronics module 12 may also be electrically coupled to analyte sensor 10, such that the components may be electromechanically coupled to one another. Analyte sensor electronics module 12 may include hardware, firmware, and/or software that enables measurement and/or estimation of levels of the analyte in a host/user via analyte sensor 10 (e.g., which may be/include a glucose sensor). For example, analyte sensor electronics module 12 can include one or more of a potentiostat, a power source for providing power to analyte sensor 10, other components useful for signal processing and data storage, and a telemetry module for transmitting data from the sensor electronics module to one or more display devices. Electronics can be affixed to a printed circuit board (PCB) within analyte sensor system 8, or platform or the like, and can take a variety of forms. For example, the electronics can take the form of an integrated circuit (IC), such as an Application-Specific Integrated Circuit (ASIC), a microcontroller, a processor, and/or a state machine.

Analyte sensor electronics module 12 may include sensor electronics that are configured to process sensor information, such as sensor data, and generate transformed sensor data and displayable sensor information. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

With further reference to FIG. 1, display devices 110, 120, 130, and/or 140 can be configured for displaying (and/or alarming) displayable sensor information that may be transmitted by analyte sensor electronics module 12 (e.g., in a customized data package that is transmitted to the display devices based on their respective preferences). Each of display devices 110, 120, 130, or 140 can (respectively) include a display such as touchscreen display 112, 122, 132,/or 142 for displaying sensor information and/or analyte data to a user and/or receiving inputs from the user. For example, a graphical user interface (GUI) may be presented to the user for such purposes. In embodiments, the display devices may include other types of user interfaces such as voice user interface instead of or in addition to a touchscreen display for communicating sensor information to the user of the display device and/or receiving user inputs. In embodiments, one, some, or all of display devices 110, 120, 130, 140 may be configured to display or otherwise communicate the sensor information as it is communicated from analyte sensor electronics module 12 (e.g., in a data package that is transmitted to respective display devices), without any additional prospective processing required for calibration and/or real-time display of the sensor data.

The plurality of display devices 110, 120, 130, 140 depicted in FIG. 1 may include a custom display device, for example, analyte display device 110, specially designed for displaying certain types of displayable sensor information associated with analyte data received from analyte sensor electronics module 12 (e.g., a numerical value and/or an arrow, in embodiments). In embodiments, one of the plurality of display devices 110, 120, 130, 140 includes a smartphone, such as a mobile phone, based on an Android, IOS, or other operating system, and configured to display a graphical representation of the continuous sensor data (e.g., including current and/or historic data).

As further illustrated in FIG. 1 and mentioned above, system 100 may also include WAP 138 that may be used to couple one or more of analyte sensor system 8, the plurality display devices 110, 120, 130, 140 etc., server system 134, and partner devices 136 to one another. For example, WAP 138 may provide WiFi and/or cellular or other wireless connectivity within system 100. Near Field Communication (NFC) may also be used among devices of system 100 for exchanging data, as well as for performing specialized functions, e.g., waking up or powering a device or causing the device (e.g., analyte sensor electronics module 12 and/or a transmitter) to exit a lower power mode or otherwise change states and/or enter an operational mode. Server system 134 may be used to collect analyte data from analyte sensor system 8 and/or the plurality of display devices, for example, to perform analytics thereon, generate universal or individualized models for glucose levels and profiles, provide services or feedback, including from individuals or systems remotely monitoring the analyte data, and so on. Partner device(s) 136, by way of overview and example, can usually communicate (e.g., wirelessly) with analyte sensor system 8, including for authentication of partner device(s) 136 and/or analyte sensor system 8, as well as for the exchange of analyte data, medicament data, other data, and/or control signaling or the like. Partner devices 136 may include a passive device in example embodiments of the disclosure. One example of partner device 136 may be an insulin pump for administering insulin to a user in response and/or according to an analyte level of the user as measured/approximated using analyte sensor system 8. For a variety of reasons, it may be desirable for such an insulin pump to receive and track glucose values transmitted from analyte sensor system 8 (with reference to FIG. 1 for example). One example reason for this is to provide the insulin pump a capability to suspend/activate/control insulin administration to the user based on the user's glucose value being below/above a threshold value.

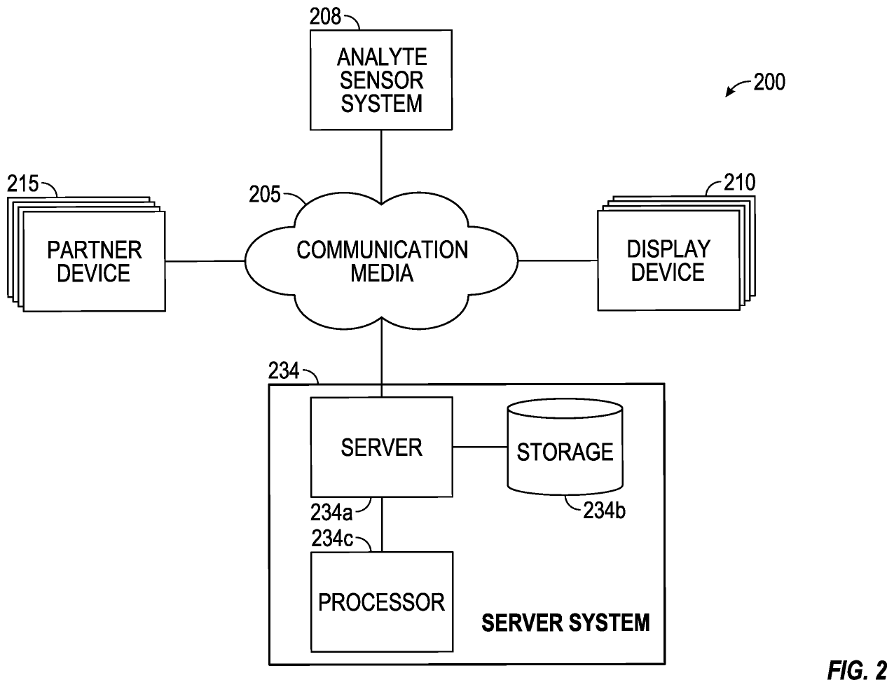

Referring now to FIG. 2, system 200 is depicted. System 200 may be used in connection with implementing embodiments of the disclosed systems, methods, apparatuses, and/or devices, including, for example, aspects described above in connection with FIG. 1. By way of example, various below-described components of FIG. 2 may be used to provide wireless communication of analyte (e.g., glucose) data, for example among/between analyte sensor system 208, display devices 210, partner devices 215, and/or one or more server systems 234, and so on. In some cases, analyte sensor system 208 illustrated in FIG. 2 may be an example of the analyte sensor system 8 illustrated in FIG. 1. Additionally, in some cases, the display devices 210 illustrated in FIG. 2 may be examples of the display devices 110, 120, 130, and 140 illustrated in FIG. 1. Additionally, in some cases, partner devices 215 illustrated in FIG. 2 may be examples of the partner device 136 illustrated in FIG. 1.

As shown in FIG. 2, system 200 may include analyte sensor system 208, one or more display devices 210, and/or one or more partner devices 215. Additionally, in the illustrated embodiment, system 200 includes server system 234, which can in turn includes server 234a coupled to processor 234c and storage 234b. Analyte sensor system 208 may be coupled to display devices 210, partner devices 215, and/or server system 234 via communication media 205. Some details of the processing, gathering, and exchanging of data, and/or executing actions (e.g., providing medicaments or related instructions) by analyte sensor system 208, partner devices 215, and/or display device 210, etc., are provided below.

Analyte sensor system 208, display devices 210, and/or partner devices 215 may exchange messaging (e.g., control signaling) via communication media 205, and communication media 205 may also be used to deliver analyte data to display devices 210, partner devices 215, and/or server system 234. As alluded to above, display devices 210 may include a variety of electronic computing devices, such as, for example, a smartphone, tablet, laptop, wearable device, etc. Display devices 210 may also include analyte display device 110 that may be customized for the display and conveyance of analyte data and related notifications etc. Partner devices 215 may include medical devices, such as an insulin pump or pen, connectable devices, such as a smart fridge or mirror, key fob, and other devices.

In embodiments, communication media 205 may be based on one or more wireless communication protocols, such as for example Bluetooth, Bluetooth Low Energy (BLE), ZigBee, WiFi, IEEE 802.11 protocols, Infrared (IR), Radio Frequency (RF), 2G, 3G, 4G, 5G, etc., and/or wired protocols and media. It will also be appreciated upon studying the present disclosure that communication media can be implemented as one or more communication links, including in some cases, separate links, between the components of system 200, whether or not such links are explicitly shown in FIG. 2 or referred to in connection therewith. By way of illustration, analyte sensor system 208 may be coupled to display device 210 via a first link of communication media 205 using BLE, while display device 210 may be coupled to server system 234 by a second link of communication media 205 using a cellular communication protocol (e.g., 4G LTE/5G and the like). In embodiments, a BLE signal may be temporarily attenuated to minimize data interceptions. For example, attenuation of a BLE signal through hardware or firmware design may occur temporarily during moments of data exchange (e.g., pairing).

In embodiments, the elements of system 200 may be used to perform operations of various processes described herein and/or may be used to execute various operations and/or features described herein with regard to one or more disclosed systems and/or methods. Upon studying the present disclosure, one of skill in the art will appreciate that system 200 may include single or multiple analyte sensor systems 208, communication media 205, and/or server systems 234.

As mentioned, communication media 205 may be used to connect or communicatively couple analyte sensor system 208, display devices 210, partner devices 215, and/or server system 234 to one another or to a network. Communication media 205 may be implemented in a variety of forms. For example, communication media 205 may include one or more of an Internet connection, such as a local area network (LAN), a person area network (PAN), a wide area network (WAN), a fiber optic network, internet over power lines, a hard-wired connection (e.g., a bus), DSL, and the like, or any other kind of network connection or communicative coupling. Communication media 205 may be implemented using any combination of routers, cables, modems, switches, fiber optics, wires, radio (e.g., microwave/RF, AM, FM links etc.), and the like. Further, communication media 205 may be implemented using various wireless standards, such as Bluetooth®, BLE, Wi-Fi, IEEE 802.11, 3GPP standards (e.g., 2G GSM/GPRS/EDGE, 3G UMTS/CDMA2000, or 4G LTE/LTE-A/LTE-U, 5G, or subsequent generation), etc. Upon reading the present disclosure, one of skill in the art will recognize other ways to implement communication media 205 for communications purposes and will also recognize that communication media 205 may be used to implement features of the present disclosure using as of yet undeveloped communications protocols that may be deployed in the future.

Further referencing FIG. 2, server 234a may receive, collect, and/or monitor information, including analyte data, medicament data, and related information, from analyte sensor system 208, partner devices 215 and/or display devices 210, such as input responsive to the analyte data or medicament data, or input received in connection with an analyte monitoring application running on analyte sensor system 208 or display device 210, or a medicament delivery application running on display device 210 or partner device 215. As such, server 234a may receive, collect, and/or monitor information from partner devices 215, such as, for example, information related to the provision of medicaments to a user and/or information regarding the operation of one or more partner devices 215. Server 234a may also receive, collect, and/or monitor information regarding a user of analyte sensor system 208, display devices 210, and/or partner devices 215.

In embodiments, server 234a may be adapted to receive such information via communication media 205. This information may be stored in storage 234b and may be processed by processor 234c. For example, processor 234c may include an analytics engine capable of performing analytics on information that server 234a has collected, received, etc. via communication media 205. In embodiments, server 234a, storage 234b, and/or processor 234c may be implemented as a distributed computing network, such as a Hadoop® network, or as a relational database or the like. The aforementioned information may then be processed at server 234a such that services may be provided to analyte sensor system 208, display devices 210, partner devices 215, and/or a user(s) thereof. For example, such services may include diabetes management feedback for the user.

In embodiments, a database may be implemented in server system 234 that may pair user accounts to one or more analyte sensor systems 208 using communication media 205. Based on, for example, an expected lifetime of individual components or one or more groups of components of analyte sensor system 208, or analyte sensor system 208 as a whole, and/or based on diagnostic feedback received by analyte sensor system 208, server system 234 may be able to determine if a given analyte sensor system 208 or component or group(s) of components thereof is expired or passed its useful life. A user may receive an indication, notification, alert, or warning, for example, on display device 210 and/or through analyte sensor system 208, from server system 234, that analyte sensor system 208 or a component or group(s) of components thereof has expired or passed its useful life or will do so soon or within a given amount of time. In embodiments, a user may receive an indication, notification, alert, or warning on display device 210 from server system 234 about the expected lifetime of analyte sensor system 208 or a component or group(s) of components thereof.

Server 234a may include, for example, an Internet server, a router, a desktop or laptop computer, a smartphone, a tablet, a processor, a module, or the like, and may be implemented in various forms, including, for example, an integrated circuit or collection thereof, a printed circuit board or collection thereof, or in a discrete housing/package/rack or multiple of the same. In embodiments, server 234a at least partially directs communications made over communication media 205. Such communications may include the delivery of analyte data, medicament data, and/or messaging related thereto (e.g., advertisement, authentication, command, or other messaging). For example, server 234a may process and exchange messages between and/or among analyte sensor system 208, display devices 210, and/or partner devices 215 related to frequency bands, timing of transmissions, security/encryption, alarms, alerts, notifications, and so on. Server 234a may update information stored on analyte sensor system 208, partner devices 215, and/or display devices 210, for example, by delivering applications thereto or updating the same, and/or by reconfiguring system parameters or other settings of analyte sensor system 208, partner devices 215, and/or display devices 210. Server 234a may send/receive information to/from analyte sensor system 208, partner devices 215, and/or display devices 210 in real time, periodically, sporadically, or on an event-drive basis. Further, server 234a may implement cloud computing capabilities for analyte sensor system 208, partner devices 215, and/or display devices 210.

With the above description of aspects of the presently disclosed systems and methods for wireless communication of analyte data, examples of some specific features of the present disclosure will now be provided. It will be appreciated by one of skill in the art upon studying the present disclosure that these features may be implemented using aspects and/or combinations of aspects of the example configurations described above, whether or not explicit reference is made to the same.

### Analyte Data

Referring back to FIG. 1, as mentioned above, in embodiments, analyte sensor system 8 is provided for measurement of an analyte in a host or user. By way of an overview and an example, analyte sensor system 8 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data to remote devices (e.g., display devices 110, 120, 130, 140, partner devices 136, and/or server system 134).

Analyte sensor system 8 may include: analyte sensor 10 configured to measure a concentration or level of the analyte in the host, and analyte sensor electronics module 12 that is typically physically connected to analyte sensor 10 before analyte sensor 10 is implanted in a user. In some cases, the analyte sensor 10 may be a multi-analyte sensor capable for measuring multiple different types of analytes, such as glucose, lactate, potassium, and the like. In embodiments, analyte sensor electronics module 12 includes electronics configured to process a data stream associated with an analyte concentration measured by analyte sensor 10, in order to generate sensor information that includes raw sensor data, transformed sensor data, and/or any other sensor data, for example. Analyte sensor electronics module 12 may further be configured to generate analyte sensor information that is customized for respective display devices 110, 120, 130, 140, partner devices 136, and/or server system 134. Analyte sensor electronics module 12 may further be configured such that different devices may receive different sensor information and may further be configured to wirelessly transmit sensor information to such display devices 110, 120, 130, 140, partner devices 136, and/or server system 134.

The term “analyte” as used herein is a broad term and is to be given its ordinary and customary meaning to a person of ordinary skill in the art (and is not to be limited to a special or customized meaning), and furthermore refers without limitation to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensor heads, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-hydroxy-cholic acid; cortisol; creatine kinase; creatine kinase MM isoenzyme; cyclosporin A; d-penicillamine; de-cthylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1,); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; elements; trace transferring; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

### Analyte Sensor System

As described to above with reference to FIG. 1, in some embodiments, analyte sensor 10 includes a continuous glucose sensor, for example, a subcutaneous, transdermal (e.g., transcutaneous), or intravascular device. In embodiments, such a sensor or device can continuously measure and analyze glucose measurements in the interstitial fluid, blood samples, etc., depending on whether the device is subcutaneous, transdermal, or intravascular. Analyte sensor 10 can use any method of analyte measurement, including for example glucose-measurement, including enzymatic, chemical, physical, electrochemical, spectrophotometric, polarimetric, calorimetric, iontophoretic, radiometric, immunochemical, and the like.

In embodiments where analyte sensor 10 is a glucose sensor, analyte sensor 10 can use any method, including invasive, minimally invasive, and non-invasive sensing techniques (e.g., fluorescence monitoring), or the like, to provide a data stream indicative of the concentration of glucose in a host. The data stream may be a raw data signal, which may be converted into a calibrated and/or filtered data stream that can be used to provide a useful value of glucose to a user, such as a patient or a caretaker (e.g., a parent, a relative, a guardian, a teacher, a doctor, a nurse, or any other individual that has an interest in the wellbeing of the host).

A glucose sensor can be any device capable of measuring the concentration of glucose. According to one example embodiment described below, an implantable glucose sensor may be used. However, it should be understood that the devices and methods described herein can be applied to any device capable of detecting a concentration of an analyte, glucose for example, and providing an output signal that represents the concentration of the analyte, again glucose for example (e.g., as a form of analyte data).

In embodiments, analyte sensor 10 is an implantable glucose sensor, such as described with reference to U.S. Pat. No. 6,001,067 and U.S. Patent Publication No. US-2005-0027463-A1. In embodiments, analyte sensor 10 is a transcutaneous glucose sensor, such as described with reference to U.S. Patent Publication No. US-2006-0020187-A1. In embodiments, analyte sensor 10 is configured to be implanted in a host vessel or extracorporeally, such as is described in U.S. Patent Publication No. US-2007-0027385-A1, co-pending U.S. Patent Publication No. US-2008-0119703-A1 filed Oct. 4, 2006, U.S. Patent Publication No. US-2008-0108942-A1 filed on Mar. 26, 2007, and U.S. Patent Application No. US-2007-0197890-A1 filed on Feb. 14, 2007. In embodiments, the continuous glucose sensor includes a transcutaneous sensor such as described in U.S. Pat. No. 6,565,509 to Say et al., for example. In embodiments, analyte sensor 10 is a continuous glucose sensor that includes a subcutaneous sensor such as described with reference to U.S. Pat. No. 6,579,690 to Bonnecaze et al. or U.S. Pat. No. 6,484,046 to Say et al., for example. In embodiments, the continuous glucose sensor includes a refillable subcutaneous sensor such as described with reference to U.S. Pat. No. 6,512,939 to Colvin et al., for example. The continuous glucose sensor may include an intravascular sensor such as described with reference to U.S. Pat. No. 6,477,395 to Schulman et al., for example. The continuous glucose sensor may include an intravascular sensor such as described with reference to U.S. Pat. No. 6,424,847 to Mastrototaro et al., for example.

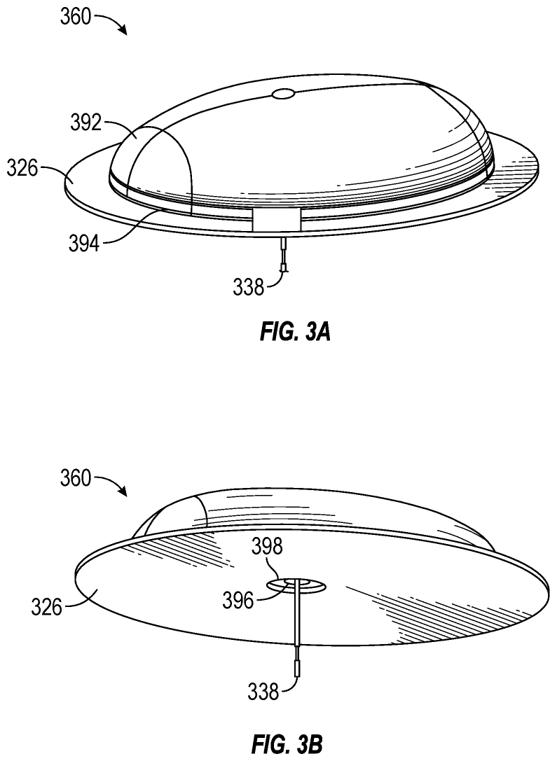

FIG. 3A illustrates a perspective view of an on-skin sensor assembly 360 that may be used in connection with an analyte sensor system 8, 208, in accordance with some embodiments. For example, on-skin sensor assembly 360 may include analyte sensor system 8, with reference by way of example to FIG. 1. On-skin sensor assembly 360 may include an outer housing with a first, top portion 392 and a second, bottom portion 394. In embodiments, the outer housing may include a clamshell design. On-skin sensor assembly 360 may include, for example, similar components as analyte sensor electronics module 12 described above in connection with FIG. 1, for example, a potentiostat, a power source for providing power to analyte sensor 10, signal processing components, data storage components, and a communication module (e.g., a telemetry module) for one-way or two-way data communication, a printed circuit board (PCB), an integrated circuit (IC), an Application-Specific Integrated Circuit (ASIC), a microcontroller, and/or a processor.

As shown in FIG. 3A, the outer housing may feature a generally oblong shape. The outer housing may further include aperture 396 disposed substantially through a center portion of outer housing and adapted for sensor 338 and needle insertion through a bottom of on-skin sensor assembly 360. In embodiments, aperture 396 may be a channel or elongated slot. On-skin sensor assembly 360 may further include an adhesive patch 326 configured to secure on-skin sensor assembly 360 to skin of the host. In embodiments, adhesive patch 326 may include an adhesive suitable for skin adhesion, for example a pressure sensitive adhesive (e.g., acrylic, rubber-based, or other suitable type) bonded to a carrier substrate (e.g., spun lace polyester, polyurethane film, or other suitable type) for skin attachment, though any suitable type of adhesive is also contemplated. As shown, adhesive patch 326 may feature an aperture 398 aligned with aperture 396 such that sensor 338 may pass through a bottom of on-skin sensor assembly 360 and through adhesive patch 326.

FIG. 3B illustrates a bottom perspective view of on-skin sensor assembly 360 of FIG. 3A. FIG. 3B further illustrates aperture 396 disposed substantially in a center portion of a bottom of on-skin sensor assembly 360, and aperture 398, both adapted for sensor 338 and needle insertion.

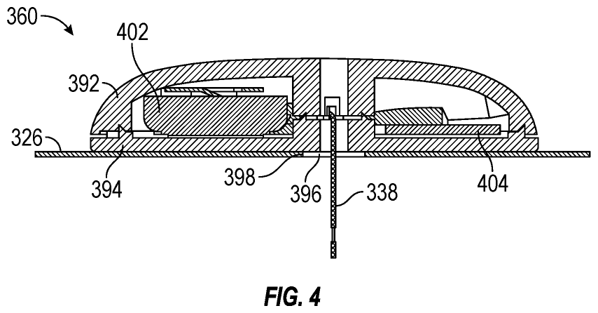

FIG. 4 illustrates a cross-sectional view of on-skin sensor assembly 360 of FIGS. 3A and 3B. FIG. 4 illustrates first, top portion 392 and second, bottom portion 394 of the outer housing, adhesive patch 326, aperture 396 in the center portion of on-skin sensor assembly 360, aperture 398 in the center portion of adhesive patch 326, and sensor 338 passing through aperture 396. The electronics unit, previously described in connection with FIG. 3A, may further include circuit board 404 and battery 402 configured to provide power to at least circuit board 404.

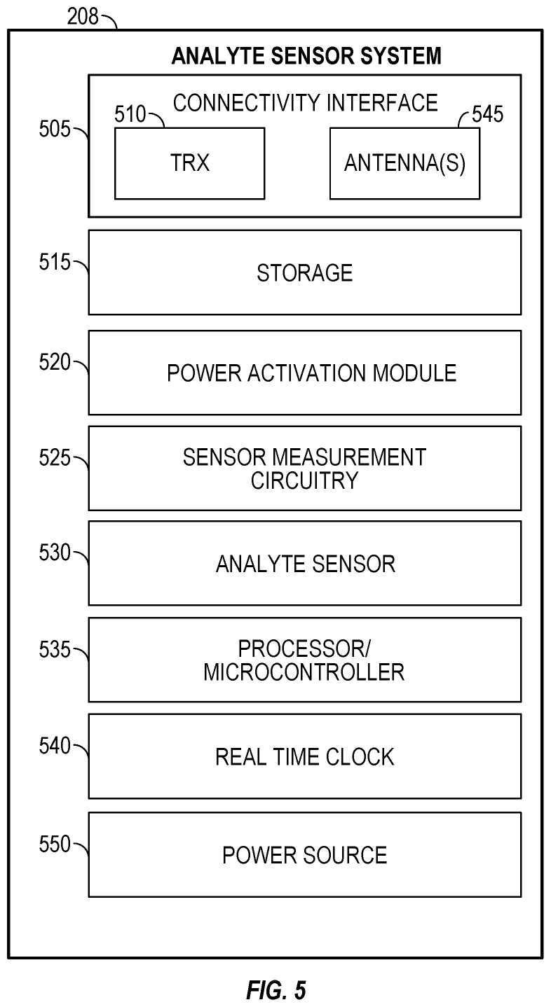

Turning now to FIG. 5, a more detailed functional block diagram of analyte sensor system 208 (discussed above, for example, in connection with FIGS. 1 and 2) is provided. As noted above, the analyte sensor system 208 may be an example of the analyte sensor system 8 illustrated in FIG. 1. As shown in FIG. 5, analyte sensor system 208 may include analyte sensor 530 (e.g., which may be an example of the analyte sensor 10 illustrated in FIG. 1) coupled to sensor measurement circuitry 525 for processing and managing analyte data measured by the analyte sensor 530. Sensor measurement circuitry 525 may be coupled to processor/microcontroller 535 (e.g., which may be part of analyte sensor electronics module 12 in FIG. 1). In some embodiments, processor/microcontroller 535 may perform part or all of the functions of sensor measurement circuitry 525 for obtaining and processing sensor measurement values (e.g., analyte data) from analyte sensor 530.

Processor/microcontroller 535 may be further coupled to a radio unit or transceiver 510 (e.g., which may be part of analyte sensor electronics module 12 in FIG. 1). In some embodiments, the processor/microcontroller 535 may be configured to provide sending sensor data, such as the analyte data, and other data to the transceiver 510 for transmission to an external device, such as display device 210 (referencing FIG. 2 by way of example). The transceiver 510 may also be configured to receive, from the external device, control information including requests for certain information and commands to perform certain actions. In some cases, the transceiver 510 may include logic or circuitry for communicating (e.g., transmitting and receiving) using different communication protocols, such as BLUETOOTH, BLUETOOTH Low Energy (BLE), near-field communication (NFC), WiFi, Third Generation Partnership Project (3GPP)-based wireless communication protocols, and other wireless communication protocols. In some embodiments, the transceiver 510 may be coupled to an antenna system 545 associated with the connectivity interface 505, allowing the analyte sensor system 208 to wirelessly transmit and receive data. For example, the transceiver 510 may be configured to output data, such as the analyte data for wireless transmission via one or more antennas of the antenna system 545 or may be configured to obtain data that is wirelessly received via the one or more antennas of the antenna system 545.

As shown, analyte sensor system 208 may also include a power activation module 520. Power activation module 520 may use or include logic circuitry configured to perform functionalities as described herein with respect to detecting triggering events, such as the presence or absence of a magnetic field, and enabling activation of analyte sensor system 8, 208 based on the triggering events. Additional details regarding the power activation module 520 are discussed further herein.

Analyte sensor system 208, in example implementations, gathers analyte data using analyte sensor 530 and transmits the same or a derivative thereof to display device 310, partner device 315, and/or server system 334. Data points regarding analyte values may be gathered and transmitted over the life of analyte sensor 530. New measurements and/or related information may be transmitted often enough for a remote device/individual to adequately monitor analyte (e.g., glucose) levels.

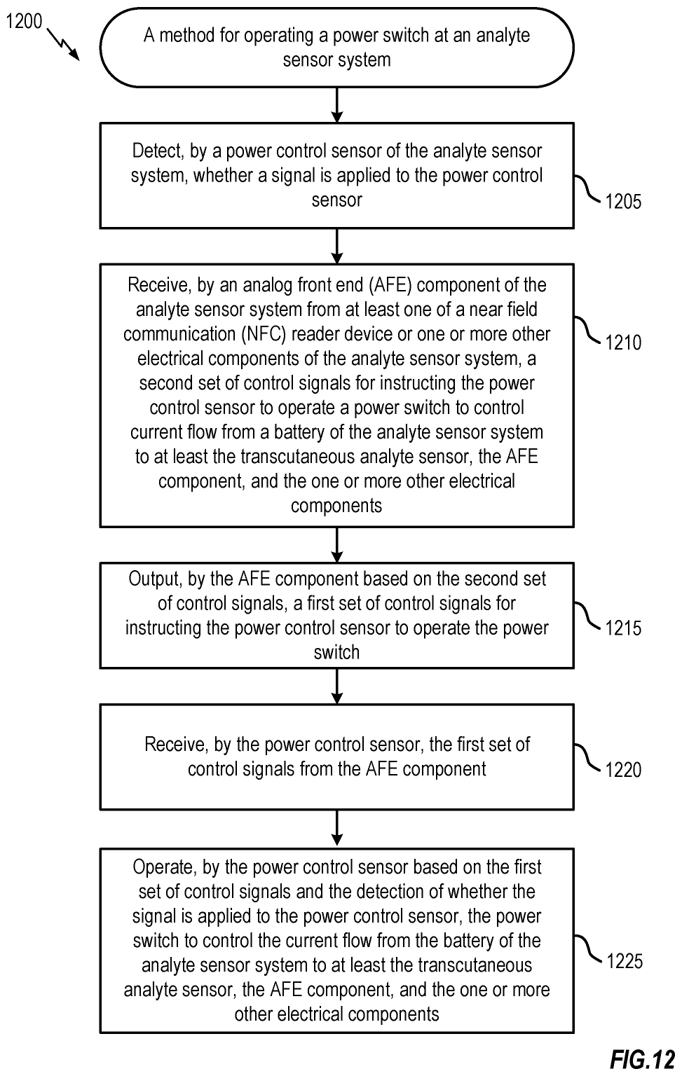

Analyte sensor system 208 may further include storage 515 (e.g., which may be part of analyte sensor electronics module 12 in FIG. 1) and real time clock (RTC) 540 (e.g., which may be part of analyte sensor electronics module 12 in FIG. 1), for storing and tracking sensor data and other data. For example, in some embodiments, analyte data measured by the analyte sensor 530 may be stored in storage 515 before being processed by the processor/microcontroller 535 and transmitted using the transceiver 510 and antenna system 545. In some cases, the storage 515 may also include instructions that, when executed by the processor/microcontroller 535, cause the analyte sensor system 208 to perform one or more actions as described herein, such as for gathering analyte data and transmitting it using the transceiver 510 and antenna system 545. In some embodiments, the storage 515 may also include instructions that, when executed by the processor/microcontroller 535, cause the analyte sensor system 208 to perform method 1200 described with respect to FIG. 12 for operating a power switch in the analyte sensor system 208.

Additionally, as shown, the analyte sensor system 208 includes a power source 550, such as a battery, configured to provide power to one or more other components of the analyte sensor system 208, such as the transceiver 510, storage 515, sensor measurement circuitry 525, analyte sensor 530, processor/microcontroller 534, real time clock 540, and the antenna system 545.

It is to be appreciated that some details of the processing, gathering, and exchanging data by analyte sensor system 208, partner devices 215, and/or display device 210 etc. are provided elsewhere herein. It will be appreciated upon studying the present disclosure that analyte sensor system 208 may contain several like components that are described with respect to FIG. 1 or 2, at least for some embodiments herein. The details and uses of such like components may therefore be understood vis-a-vis analyte sensor system 208 even if not expressly described here with reference to FIG. 5.

### Aspects Related to NFC-Based Power Control for an Analyte Sensor System

Patients with diabetes may benefit from real-time diabetes management guidance that is determined based on a physiological state of the patient. In certain cases, the physiological state of the patient is determined using diagnostics systems, such as an analyte sensor system (e.g., analyte sensor system 8 and/or analyte sensor system 208). In some embodiments, analyte sensor system 208 may be configured to measure analyte levels and inform a patient about the identification and/or prediction of adverse events, such as hyperglycemia and hypoglycemia. Additionally, the analyte sensor system 208 may be configured to help inform the type of guidance provided to the patient in response to these adverse glycemic events.

For example, the analyte sensor system 208 may be worn by a patient and configured to continuously measure the patient's analyte levels over time using a continuous analyte sensor, such as the analyte sensor 530. The measured analyte levels may then be processed by the analyte sensor system 208 (e.g., by the processor/microcontroller 535) to identify and/or predict adverse events, and/or to provide guidance to the patient for treatment and or actions to abate or prevent the occurrence of such adverse events. To perform and process these measurements, the analyte sensor system 208 may be equipped with a power source (e.g., power source 550), such as a battery. When the analyte sensor system 208 is powered on, power from the battery may be consumed by different components of the analyte sensor system 208, such as one or more sensors (e.g., sensor measurement circuitry 525, analyte sensor 530, etc.), microprocessors (e.g., processor/microcontroller 535), transmitters (e.g., connectivity interface 505), and the like. Because the amount of power that may be stored within a battery is limited, power consumption in analyte sensor systems is a primary concern.

To help conserve battery power, the analyte sensor system 208 may be equipped with a power activation module, such as the power activation module 520. The power activation module 520 may be designed to maintain the analyte sensor system 208 in an OFF state or a low power state in the absence of a trigger event and to transition the analyte sensor system 208 into an ON state or a high power state at an occurrence of the trigger event. In some embodiments, the ON state or high power state may be associated with an operational mode of the analyte sensor system 208 in which the analyte sensor system 208 performs analyte measurements and provides these measurements, or data indicating these measurements, to a display device or receiver. In some embodiments, the power activation module 520 may include a power control sensor, such as tunnel magnetoresistance (TMR) sensor, configured to maintain the analyte sensor system 208 in the OFF state in the absence of a trigger event. For example, the TMR sensor may be configured to output a discrete signal in the presence of an applied magnetic field. This discrete signal may, in turn, be used to control the flow of current from a battery of the analyte sensor system 208 and, consequently, whether the analyte sensor system 208 is powered ON or OFF.

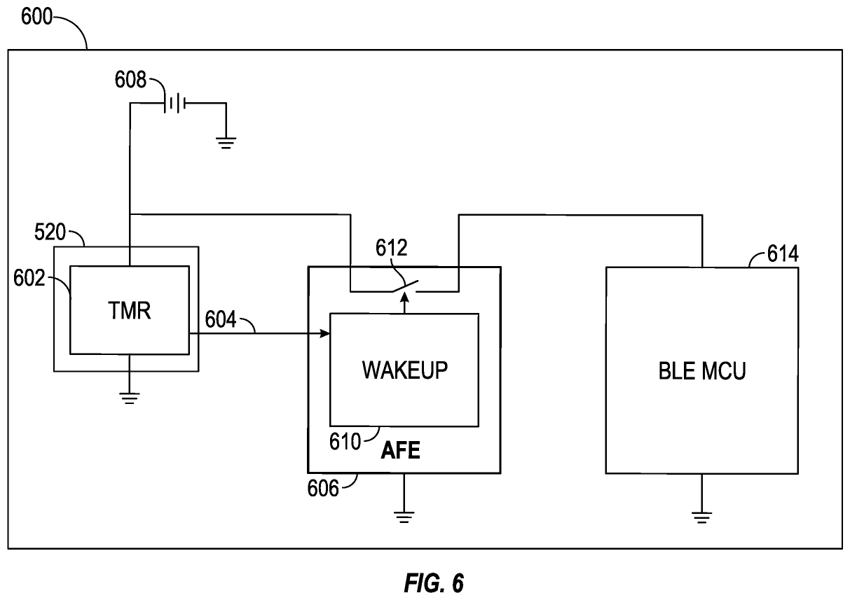

FIG. 6 illustrates a simplified block diagram of the analyte sensor system 600 including the power activation module 520 of FIG. 5. As shown, the power activation module 520 includes a TMR sensor 602. Further, as shown at, an output signal 604 of the TMR sensor 602 may be provided to an analog front end (AFE) component 606 that is continually powered by battery 608 and configured to sense the output of the TMR sensor 602. Accordingly, when the output signal of the TMR sensor 602 is low (e.g., a logical zero), a wake-up component 610 within the AFE component 606 is configured to maintain a switch 612 in the AFE component 606 in an open position, preventing current flow from the battery 608 to a Bluetooth low energy (BLE) micro control unit (MCU) 614 of the analyte sensor system 600 and maintaining the analyte sensor system 600 in the OFF state.

In some embodiments, when a magnetic field is not applied to or is far from the TMR sensor 602, the output signal of the TMR sensor 602 may be configured to be high (e.g., a logical one). Accordingly, when the magnetic field is removed from the TMR sensor 602, the wake-up component 610 of the AFE component 606 senses the high output signal 604 from the TMR sensor 602 and closes the switch 612, allowing for current to flow from the battery 608 to the BLE MCU 614 and allowing the analyte sensor system 600 to transition to the ON state.

For purposes of conserving energy of the battery 608, it may be desirable to maintain the analyte sensor system 600 in the OFF state when not being used or not being worn by a patient, such as when the analyte sensor system 600 is still in its packaging and being stored or shipped. Thereafter, the analyte sensor system 600 may be transitioned into the ON state when removed from the packaging and actively being used or worn by the patient. In some embodiments, to achieve the transition from the OFF state (e.g., while not being used or worn) to the ON state (e.g., while being used or worn), a magnet may be included within the packaging or housing of the analyte sensor system 600. While the continuous glucose monitor resides in its packaging or housing, the magnet applies a continuous magnetic field to the TMR sensor 602 included within the power activation module 520, maintaining the analyte sensor system 600 in the OFF state to conserve battery power. When the analyte sensor system 600 is removed from its packaging or housing, the magnetic field may no longer be applied to the TMR sensor 602, allowing the current from the battery 608 to flow and forcing the analyte sensor system 600 into the ON state.

While power consumption in analyte sensor system 600 is at its highest when the analyte sensor system 600 is powered on (e.g., in the ON state), power may still be consumed or dissipated by the analyte sensor system 600 when in the analyte sensor system 600 is in the OFF state. This power consumption is the result of the wake-up component 610 in the AFE component 606 having to be continually powered on in order to sense whether the output signal of the TMR sensor 602 is high or low. Albeit comparatively low to the ON state, the power consumption in the OFF state may still be problematic. For example, when the analyte sensor system 600 is stored for substantial amounts of time, this power consumption experienced in the OFF state may reduce battery power to such a level that the analyte sensor system 600 becomes unusable. Moreover, this power consumption presents a significant issue to next generation analyte sensor systems whose form factors are significantly reduced (e.g., due to competition and patient comfortability purposes), resulting in the use of smaller batteries that have less energy storage capacity.

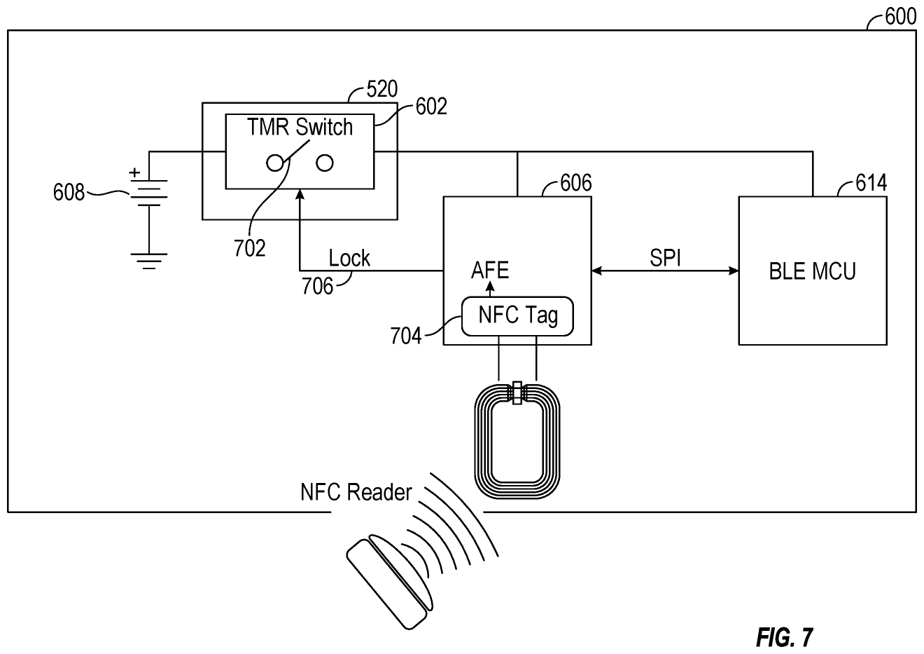

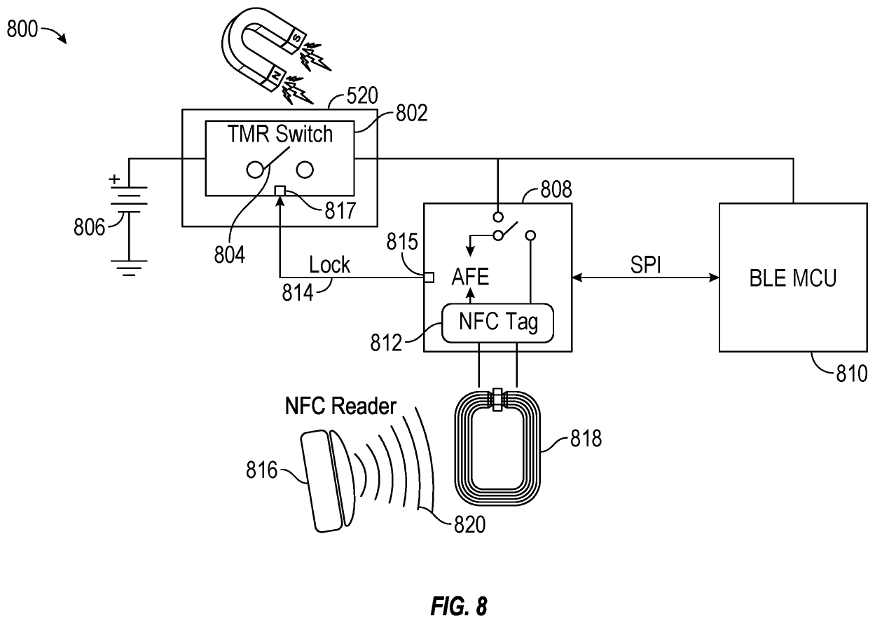

Accordingly, in some cases, rather than having the AFE component 606 be continuously powered to sense whether the TMR sensor 602 is HIGH or LOW, in next-generation analyte sensor systems, the switch 612 (e.g., that controls the current flow from the battery 608 to the BLE MCU 614) may be moved out of the AFE component 606 and instead be replaced by a switch in the TMR sensor 602. For example, as shown in FIG. 7, the TMR sensor 602 of the power activation module 520 may include a power switch 702 that is configured to control the flow of current from the battery 608 to the AFE component 606 and the BLE MCU 614. For example, prior to being deployed by a patient, when a magnetic field is applied to or is near the TMR sensor 602, the power switch 702 in the TMR sensor 602 may be maintained in an open configuration, as shown, preventing current from flowing from the battery 608 to the AFE component 606 and BLE MCU 614 and allowing the analyte sensor system 208 to be maintained in the OFF state. Conversely, when the magnetic field is removed from or not applied to the TMR sensor 602, the power switch 702 in the TMR sensor 602 may be closed, allowing the current to flow from the battery 608 to the AFE component 606 and BLE MCU 614. Notably, because the current flow is controlled by the power switch 702 in the TMR sensor 602 of FIG. 7, the AFE component 606 does not need to be continually powered in order to sense the output of the TMR sensor 602, thereby conserving power of the battery 608.

Additionally, as shown, once deployed and the analyte sensor system 208 is powered on, the AFE component 606 may be configured to output a LOCK signal 706 to a control pin or LOCK pin of the TMR sensor 602. The LOCK signal 706 may function to “lock” the power switch 702 in a closed position, preventing the analyte sensor system 208 from unintentionally being powered down if a magnetic field were to be applied to the TMR sensor 602 after deployment of the analyte sensor system 600.

While the techniques discussed above related to controlling the current flow from the battery 608 via the power switch 702 in the TMR sensor 602 may help to reduce power consumption after the analyte sensor system 600 is finished being manufactured and is put into storage prior to use by a patient, power consumption may still be an issue for next generation analyte sensors during a manufacturing process of these next-generation analyte sensor systems. For example, during the manufacturing process, it may not be feasible (e.g., due to issues with complexity and cost) to continuously keep a magnet in close proximity to the TMR sensor 602 to ensure the analyte sensor system 600 is powered off.

As a result, once the battery 608 is inserted onto a printed circuit board assembly (PCBA) of the analyte sensor system 208 of FIG. 7, the AFE component 606 and the BLE MCU 614 may power up since, without a magnet in close proximity of the TMR sensor 602, the power switch 702 in the TMR sensor 602 is closed, allowing current to flow from the battery 608. Additionally, a significant amount of time may pass before the analyte sensor system 600 is finished being manufactured and is finally placed in close proximity to a magnet (e.g., included within packaging of the analyte sensor system 600). For example, a time between insertion of the battery 608 onto the PCBA and insertion of the analyte sensor system 208 within its packaging may comprise a few months. Even if the analyte sensor system 600 were to be placed into a lowest power mode of the ON state, the analyte sensor system 208 may still experience up to 10 percent power depletion of the battery 608. This is especially problematic for next generation analyte sensor systems that will be expected to do perform more advanced operations (e.g., impedance measurements, multi-analyte measurements, etc.) for a longer period of time (e.g., 15.5 days as compared to 10.5 days of existing analyte sensory systems).

One manner of addressing the issue of power consumption in next-generation analyte sensor systems that employ TMR sensors for controlling current flow from a battery, such as the analyte sensor system 600 shown in FIG. 7, may simply be to refrain from inserting the battery 608 until the analyte sensor system 600 is finished being manufactured. However, there are multiple times during the manufacturing process of the analyte sensor system 600 in which the analyte sensor system 600 is tested for proper operation. For example, as shown in FIG. 7, the AFE component 606 of the analyte sensor system 600 includes a near field communication (NFC) tag 704 that may be used to perform the testing. However, performing the testing may require the analyte sensor system 600 to be powered up, for example, by the battery 608. As a result, because these testing procedures require the analyte sensor system 600 to be powered up (e.g., by the battery 608), simply inserting the battery 608 at the end of the manufacturing process may not be feasible.

Accordingly, aspects of the present disclosure provide techniques for reducing power consumption during a manufacturing process of next generation analyte sensor systems. In some cases, these techniques may involve using an NFC/radio frequency identifier (RFID) tag included within an AFE of an analyte sensor system to control a power switch within a TMR sensor of the analyte sensor system, thereby allowing the AFE to control the current flow from the battery of the analyte sensor system. For example, as will be described in greater detail below, to reduce power consumption of a battery during the manufacturing process of the analyte sensor system, a signal may be provided to the AFE via the NFC/RFID tag that instructs the AFE to output a signal to the TMR sensor that, in turn, instructs the TMR sensor to open the power switch, cutting off current flow from the battery to the AFE and MCU of the analyte sensor system. Additionally, when testing of the analyte sensor system is to be performed, another signal may be provided to the AFE via the NFC/RFID tag that instructs the AFE to output a signal to the TMR sensor that, in turn, instructs the TMR sensor to close the power switch, allowing current to flow from the battery to the AFE and MCU of the analyte sensor system and allowing the analyte sensor system to power on.

In some cases, using an NFC/RFID tag in the AFE to control the power switch in the TMR sensor of the analyte sensor system may also be useful in scenarios other than manufacturing of the analyte sensor system. For example, there may be scenarios in which an analyte sensor system fails to operate correctly and needs to be returned to a manufacturer or retailer for failure analysis. In many cases, when the analyte sensor is returned for failure analysis, the analyte sensor system may not be received by the manufacturer or retailer for several weeks or months after which time the battery of the analyte sensor system is completely depleted. In such cases, because the battery is completely depleted, it is not possible to wirelessly communicate with the analyte sensor system in order to download necessary information in order to determine the reason for its failure to operate correctly. It may be possible to drill a hole through the analyte sensor system to get access to the battery contacts to re-power the analyte sensor system, but this process is invasive, takes a considerable amount of time (e.g., limiting the number of devices that may be analyzed), and has the potential to damage the analyte sensor system to the point it compromises the failure analysis.

FIG. 8 illustrates a simplified block diagram of the analyte sensor system 800 having an AFE component configured to operate a power switch in a power control sensor using one control pin on the power control sensor. In some embodiments, the analyte sensor system 800 may be an example of the analyte sensor system 208 illustrated in FIGS. 2 and 5. For example, as shown, the analyte sensor system 800 includes the power activation module 502, which includes a power control sensor, such a TMR sensor 802. As shown, the TMR sensor 802 includes a power switch 804 configured to control current flow from a battery 806 to an AFE component 808 and one or more other electrical components of the analyte sensor system 800, such as an MCU 810. In some embodiments, the power switch 804 may also be configured to control current flow from the battery 806 to a transcutaneous analyte sensor, such as the analyte sensor 530 illustrated in FIG. 5. In some embodiments, the MCU 810 may include one or more memories and one or more processors, such as the processor/microcontroller 535 and storage 515. In some embodiments, the one or more processors of the MCU 810 may be configured to perform or coordinate certain functions of the analyte sensor system 800, such as performance of analyte measurements by the transcutaneous analyte sensor, controlling the timing of the analyte measurements, storing the analyte measurements in the one or more memories, processing the analyte measurements (e.g., performing various calculations based on the analyte measurements) to generate analyte data indicating a level of an analyte, storing the analyte data in the one or more memories, coordinating wireless communication of analyte data to a display device, etc.

Further, as shown, the AFE component 808 includes an NFC tag 812 that may be used to perform various testing operations during a manufacturing process of the analyte sensor system 800. For example, in some cases, the NFC tag 812 of the AFE component 808 may be configured to receive signaling from an RFID reader device, such as NFC reader 816, during a manufacturing process that instructs the analyte sensor system 800 to perform certain testing procedures to ensure that the analyte sensor system 800 is operating correctly. Additionally, in some embodiments, the NFC tag 812 may include energy harvesting circuitry 818 configured to harvest energy or power from control signals 820 (e.g., including a radio frequency (RF) energy signal) received from the NFC reader 816 (e.g., a device configured to output energy signals for powering RFID-capable devices and receiving information from the RFID capable devices based on the energy signals).

As shown, the AFE component 808 may be configured to output a first set of control signals, such as LOCK signal 814, on a first control pin (e.g., LOCK pin 815) that may be received on a second control pin (e.g., LOCK pin 817) on the TMR sensor 802 and used to operate the power switch 804 of the TMR sensor 802. For example, during a manufacturing process of the analyte sensor system 800 in which the power switch 804 would normally be maintained in a closed position, allowing current flow from the battery 806 due to a magnetic field not being applied to the TMR sensor 802, the AFE component 808 may be configured to output the LOCK signal 814 to the TMR sensor 802 on a LOCK pin 815. The LOCK signal 814 may then be received on the LOCK pin 817 of the TMR sensor 802 and may instruct the TMR sensor 802 to open the power switch 804, thereby stopping the current flow from the battery 806 and conserving power.

In some embodiments, when testing of the analyte sensor system 800 is necessary, the NFC tag 812 of the AFE component 808 may receive a second set of control signals from the NFC reader 816, powering the AFE component 808 and instructing the AFE component 808 to output an additional LOCK signal 814 to the TMR sensor 802. The energy signal received from the RFID reader device may also instruct the analyte sensor system 800 to perform one or more testing procedures. This additional LOCK signal 814 may instruct the TMR sensor to close the power switch 804, thereby allowing current flow from the battery 806 to the AFE component 808 and MCU 810, powering up the analyte sensor system 800 and allowing the analyte sensor system 800 to perform the one or more testing procedures.

Because the NFC tag 812 of the AFE component 808 includes the energy harvesting circuitry, this would allow the AFE component 808 to operate the power switch 804 in the TMR sensor 802 even when the power switch 804 is open and the AFE component 808 is turned off. Further, because the TMR sensor 802 is connected to the battery 806 and is, therefore, continually powered, a state of the power switch 804 (e.g., opened or closed) may be maintained even when the AFE component 808 is powered off and not outputting the LOCK signal 814 to the TMR sensor 802. Maintaining the state of the power switch 804 may allow for electronics (e.g., AFE component 808, MCU 810, etc.) of the analyte sensor system 800 to be powered on and off as needed during manufacturing using the NFC tag 812, thereby reducing power consumption to a minimum, while still allowing the analyte sensor system 800 to remain asleep (e.g., powered down) during storage based on an applied magnetic field and allowing the analyte sensor system 800 to turn on when the magnetic field is removed during deployment.

Additionally, allowing the power switch 804 of the TMR sensor 802 to be controlled by the AFE component 808 independent from an applied or non-applied magnetic field, would allow the MCU 810 to send a command to the AFE component 808 to open the power switch 804 and to power down the analyte sensor system 800 when the MCU 810 detects a failure of the analyte sensor system 800 while in use by a user. Further, when a failure is detected, the MCU 810 may store all necessary diagnostic information about the failure in non-volatile flash memory, allowing the analyte sensor system 800 to be safely powered down. Powering down the analyte sensor system 800 upon failure would then allow for any remaining power in the battery 806 to be stored for months, ensuring the analyte sensor system 800 could be powered back up upon being received by a manufacturer or retailer. Upon reception, the manufacturer or retailer may then provide an energy signal to the NFC tag 812, powering the AFE component 808 and instructing the AFE component 808 to close the power switch 804. Once the power switch 804 is closed, the analyte sensor system 800 may again be powered on so that the manufacturer may receive the diagnostic information about the failure without the need for the lengthy and invasive drilling operation described above.

To support the dual power control described above in which the TMR sensor 802 and AFE component 808 are both capable of controlling the power switch 804, and to ensure that the analyte sensor system 800 is correctly powered ON and OFF in all use cases, functionality of the TMR sensor 802 may be modified relative to, for example, the TMR sensor 602 illustrated in FIG. 7. For example, as described above with reference to FIG. 7, the TMR sensor 602 includes a control pin controlled by the LOCK signal 706 output by the AFE component 606 that, when output, prevents re-opening the power switch 702 and turning off the analyte sensor system 208 if a magnetic field were to be reapplied to the TMR sensor 602 after deployment of the analyte sensor system 208 onto a user. In some cases, in the case of failure of the analyte sensor system 208, the LOCK signal 706 may be de-asserted and the analyte sensor system 208 could be powered off again if a magnetic field were to be re-asserted to the TMR sensor 602. However, the LOCK signal 706 would not allow the analyte sensor system 208 of FIG. 7 to be turned off without the presence of a magnetic field. As a result, the control pin and TMR sensor 602 may function as an OR gate to control the state of the power switch 702 in accordance with Table 1, below.

TABLE 1
Power Switch States of TMR Sensor

| TMR Sensor | LOCK Signal | Power Switch State |
|---|---|---|
| Magnet field present | Low | Open = Analyte sensor |
| | | system powered OFF |
| Magnet field present | High | Closed = Analyte sensor |
| | | system powered ON |
| Magnet field not present | Low | Closed = Analyte sensor |
| | | system powered ON |
| Magnet field not present | High | Closed = Analyte sensor |
| | | system powered ON |

To allow the AFE component 808 to also control the power switch 804, the TMR sensor 802 would need to be able to support additional logic states relative to Table 1. As one example, the TMR sensor 802 would need to support a logic state in which, when a magnetic field is not present, the power switch 804 is configured to be open so that the analyte sensor system 800 may be powered OFF even in the absence of a magnetic field.

However, the AFE component 808 and the TMR sensor 802 may each, respectively, only have one control pin for outputting and receiving the LOCK signal, which may present an issue with supporting these additional logic states. One manner of helping to resolve this issue is to modify the manner in which the LOCK pin 817 of the TMR sensor 802 functions so as to be sensitive to transitions of the control signal (e.g., a transition from a high control signal to a low control signal) received on the LOCK pin 817. Table 2 illustrates power switching actions/states of the power switch 804 for NFC-based power control based on the single LOCK pin 817 of the TMR sensor 802.

TABLE 2
Power Switch States of TMR Sensor with Single LOCK Pin

| TMR sensor | LOCK pin | Power switch action/state |
|---|---|---|
| Magnetic field present | Low | Maintain state |
| Magnetic field present | High | Closed = power on |
| Magnetic field not present | Low | Maintain state (*) |
| Magnetic field not present | High | Closed = power on |
| X | High −> Low | Open = turn power off |
| Magnetic field present −> | X | Closed = turn power on |
| Magnetic field not present | | |

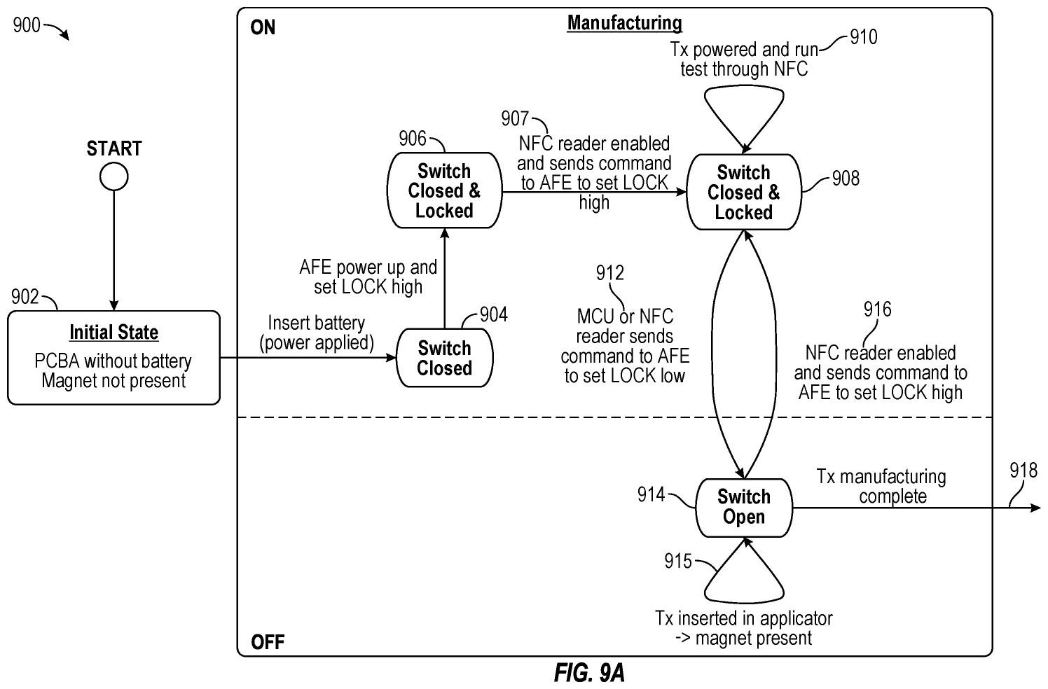

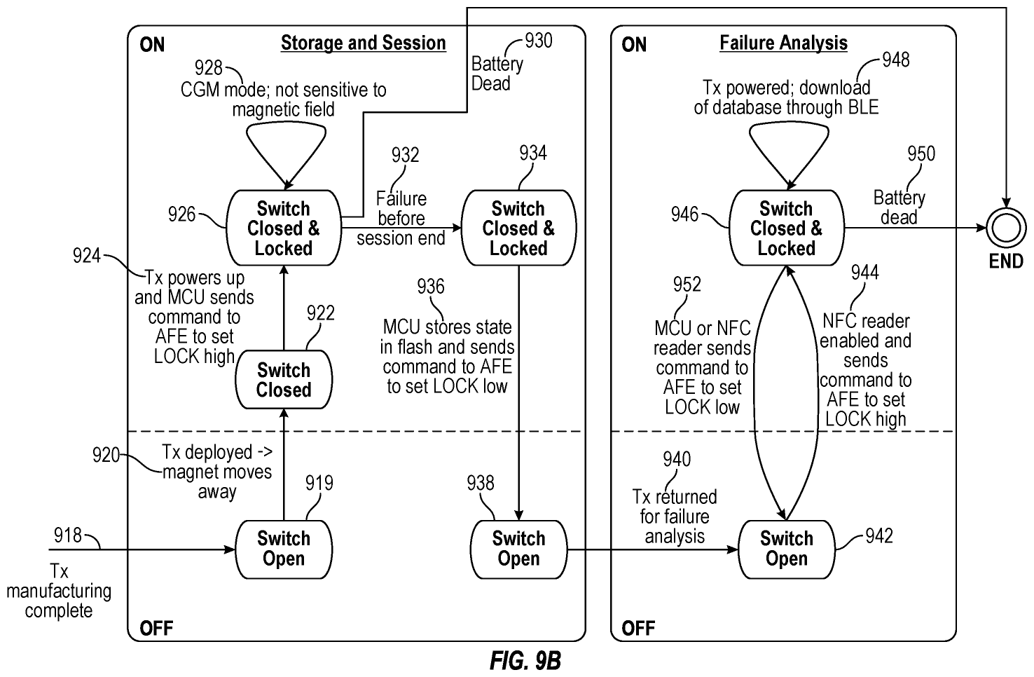

FIGS. 9A and 9B include a logic state diagram 900 associated with the power switch 804 of the TMR sensor 802, illustrating the different power switch states/actions of Table 2. As shown in FIG. 9A, the logic state diagram 900 begins in initial state 902 in which the battery 806 is not inserted on to a PCBA of the analyte sensor system 800 and a magnetic field is not applied to the TMR sensor 802. Thereafter, during a manufacturing procedure of the analyte sensor system 800, the battery 806 may be inserted into the PCBA and the logic state diagram 900 moves to state 904 in which the power switch 804 is closed and the analyte sensor system 800 is powered on, corresponding to row 2 in Table 2. After powering on, the AFE component 808 outputs a LOCK signal to the TMR sensor 802, locking the analyte sensor system 800 on, as shown in state 906 corresponding to row 3 in Table 2.

Thereafter, the analyte sensor system 800 may be configured to perform one or more testing procedures to ensure proper operation of the analyte sensor system 800. For example, as shown at 907, NFC reader 816 may send a command to the AFE component 808 to set the LOCK signal high, resulting in the power switch 804 being closed and locked in state 908 corresponding row 5 of Table 2. The analyte sensor system 800 may then perform one or more testing procedures based on one or more commands received from the NFC reader, as shown at 910.

Thereafter, as shown at 912, after the one or more testing procedures are complete, the MCU 810 or NFC reader 816 sends a command to the AFE component 808 to set the LOCK signal low. Based on the transition from the high LOCK signal to the low LOCK signal (e.g., regardless of whether a magnetic field is applied to the TMR sensor 802), the TMR sensor 802 is configured to open the power switch 804, as shown in state 914 corresponding to row 6 of Table 2. The power switch 804 may be maintained in state 914 (e.g., open state) for long periods of time during the manufacturing process in order to conserve battery power. Only when the analyte sensor system 800 needs to be tested may the power switch 804 be transitioned to a closed state and the analyte sensor system 800 powered on.

For example, as shown at 916, the NFC reader 816 may send a command to the AFE component 808 to set the LOCK signal high, resulting in the power switch 804 being closed and locked in state 908 corresponding row 5 of Table 2. The analyte sensor system 800 may then perform one or more additional testing procedures based on one or more commands received from the NFC reader 816, as shown at 910. Thereafter, as shown at 912, after the one or more additional testing procedures are complete, the MCU 810 or NFC reader 816 again sends a command to the AFE component 808 to set the LOCK signal low. Based on the transition from the high LOCK signal to the low LOCK signal, the TMR sensor 802 is configured to open the power switch 804, as shown in state 914 corresponding to row 6 of Table 2.

At some point in time, as shown at 915, the analyte sensor system 800 may be inserted into an applicator device (e.g., a device used to deploy the analyte sensor system 800 on to a user) that includes a magnet that applies a magnetic field to the TMR sensor 802. Because the LOCK signal is low, the power switch 804 remains open in state 914. In other words, state 914 is maintained when the magnetic field is applied to the TMR sensor 802 in accordance with row 4 of Table 2.

Thereafter, as shown at 918, the manufacturing procedure of the analyte sensor system 800 may be completed and the analyte sensor system 800 may be put into storage or shipped to user. The logic state diagram 900 then moves into a storage and session phase. For example, as shown in FIG. 9B, after the manufacturing procedure of the analyte sensor system is completed at 918 and while in storage or being shipped to the user, the power switch 804 may be maintained in an open position, as shown in state 919 corresponding to row 2 in Table 2. Thereafter, as shown at 920, the analyte sensor system 800 may be deployed onto the user, resulting in the magnetic field no longer being applied to the TMR sensor 802 (e.g., since after being deployed, the magnet stays in the applicator while the analyte sensor system 800 is attached to the user). Due to the magnetic field no longer being applied to the TMR sensor 802, the TMR sensor 802 closes the power switch 804, as shown in state 922 corresponding to row 7 of Table 2.

Thereafter, current from the battery 806 may begin to flow, powering on the analyte sensor system 800. Further, as shown at 924, the MCU 810 may send a command to the AFE component 808, instructing the AFE component 808 to output a high LOCK signal. Based on the LOCK signal, the power switch 804 may be maintained/locked in a closed position, as shown in state 926 corresponding to row 5 in Table 2. While the power switch 804 is closed and locked, the analyte sensor system 800 may operate in a continuous glucose monitoring (CGM) mode and may not be sensitive to changes in a magnetic field, as shown at 928. While operating in the CGM mode, the analyte sensor system 800 may perform one or more analyte measurements of the user and transmit these analyte measurements to a display device (e.g., display devices 110, 120, 130, and/or 140).

The analyte sensor system 800 may operate in the CGM mode until either the battery 806 is depleted, as shown at 930, or a failure of the analyte sensor system 800 occurs, as shown at 932. When a failure of the analyte sensor system 800 occurs, the power switch 804 may still be maintained in a closed position and locked, as shown in state 934. However, when the failure is detected, the MCU 810 may store within the non-volatile memory of the analyte sensor system 800 (e.g., storage 515 illustrated in FIG. 5) diagnostic information indicating the state of the analyte sensor system 800. The MCU 810 may also instruct the AFE component 808 to output a low LOCK signal, as shown at 936. This diagnostic information indicating the state of the analyte sensor system 800 may include information such as time of failure, type of failure, MCU register values, random access memory (RAM) data.

Thereafter, due to the transition of the LOCK signal from high to low, the TMR sensor 802 may be configured to open the power switch 804, powering off the analyte sensor system 800, as shown in state 938 corresponding to row 6 of Table 2. In some embodiments, due to the transition of the LOCK signal from high to low, the analyte sensor system 800 may be powered off even in the absence of a magnetic field being applied to the TMR sensor 802, as represented by the X in row 6 of Table 2. Powering off the analyte sensor system 800 even in the absence a magnetic field being applied to the TMR sensor 802 may allow the analyte sensor system 800 to conserve battery power, allowing the analyte sensor system 800 ample time to be shipped back to a manufacturer or retailer for failure analysis without the need for the invasive access to the battery contacts to re-power the analyte sensor system 800 described above.

After powering down, the analyte sensor system 800 may be sent back to the manufacturer or retailer for failure analysis as shown at 940. As shown, during shipment, the power switch 804 may be maintained in an open position, as shown in state 942 corresponding to row 4 of Table 2. Once received, the manufacturer or retailer may use an NFC reader 816 to transmit an energy signal to power (e.g., using energy harvesting circuitry) and instruct the AFE component 808 to output a high lock signal (e.g., using at least some energy harvested from the energy signal received from the NFC reader 816), as shown at 944.

Based the LOCK signal being set high, the TMR sensor 802 may be configured to close the power switch 804 and lock it in the closed position, as shown in state 946 corresponding to row 5 of Table 2, allowing the analyte sensor system 800 to power on again using any remaining power stored in the battery 806. While the analyte sensor system 800 is powered on, the manufacturer or retailer may download the diagnostic information indicating the state of the analyte sensor system 800 from the memory of the analyte sensor system 800, as shown at 948. The manufacturer or retailer may then perform diagnostics using the diagnostic information to determine a cause of the failure the analyte sensor system 800. In some embodiments, the manufacturer or retailer may download the diagnostic information using Bluetooth low energy (BLE), WiFi, cellular based wireless communications, a hardwire connection, etc.

In some cases, the power switch 804 may be maintained in the closed position in state 946 until the battery 806 is depleted as shown at 950. In other cases, as shown at 952, the manufacturer or retailer may decide to conserve power of the battery 806 during failure analysis and use the NFC reader 816 to instruct the AFE component 808 to output a low LOCK signal to the TMR sensor 802. Based on the transition of the LOCK signal from high to low (even in the absence of an applied magnetic field), the TMR sensor 802 may be configured to open the power switch 804 and return to state 942 corresponding to row 6 of Table 2.

The techniques presented above describe a single control pin solution (e.g., LOCK pin 817 on the TMR sensor 802) to support the additional logic states shown in Table 2. In some embodiments, however, a two control pin solution may be used. For example, to support the additional logic states, the TMR sensor 802 and the AFE component 808 may each include additional control pin, such as a force sleep (FSLP) pin.

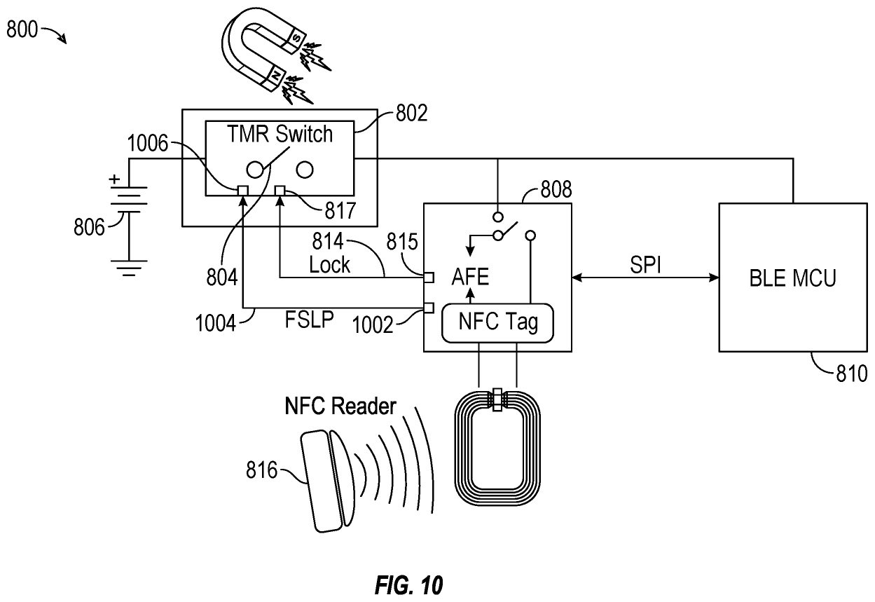

For example, FIG. 10 illustrates a simplified block diagram of the analyte sensor system 800 having the AFE component 808 configured to operate the power switch 804 in TMR sensor 802 using two control pins on the TMR sensor 802. In some embodiments, the two control pins may be used to support the additional logic states for operating the power switch 804 of the TMR sensor 802. For example, as described with respect to FIG. 8, the AFE component 808 includes a LOCK pin 815 configured for outputting the LOCK signal 814, which may be received by the TMR sensor 802 on the LOCK pin 817. Additionally, as shown in FIG. 10, the AFE component 808 may also include a FSLP pin 1002 configured for outputting a FSLP signal 1004, which may be received by the TMR sensor 802 on a FSLP pin 1006. As will be described in greater detail below, the LOCK signal 814 and the FSLP signal 1004 received on the LOCK pin 817 and the FSLP pin 1006, respectively, may be used to control different actions or states associated with the power switch 804. For example, Table 3 illustrates power switching actions/states of the power switch 804 for NFC-based power control based on two control pins (e.g., the LOCK pin 817 and the FSLP pin 1006) of the TMR sensor 802.

TABLE 3
Power Switch States of TMR Sensor with LOCK Pin and FSLP Pin

| | | FSLP | FSLP | Power switch action/ |
|---|---|---|---|---|
| TMR sensor | LOCK pin | pin | state | state |
| Magnet present | Low | X | No | Open = power off |
| Magnet present | High | X | No | Closed = power on |
| Magnet away | Low | X | No | Closed = power on |
| Magnet away | High | X | No | Closed = power on |
| X | (1) High −> | (2) 2 | No −> | Open = turn power |
| | (3) Low | pulses | Yes | off |
| X | Low | 1 pulse | Yes −> | Return to magnet |
| | | | No | control |

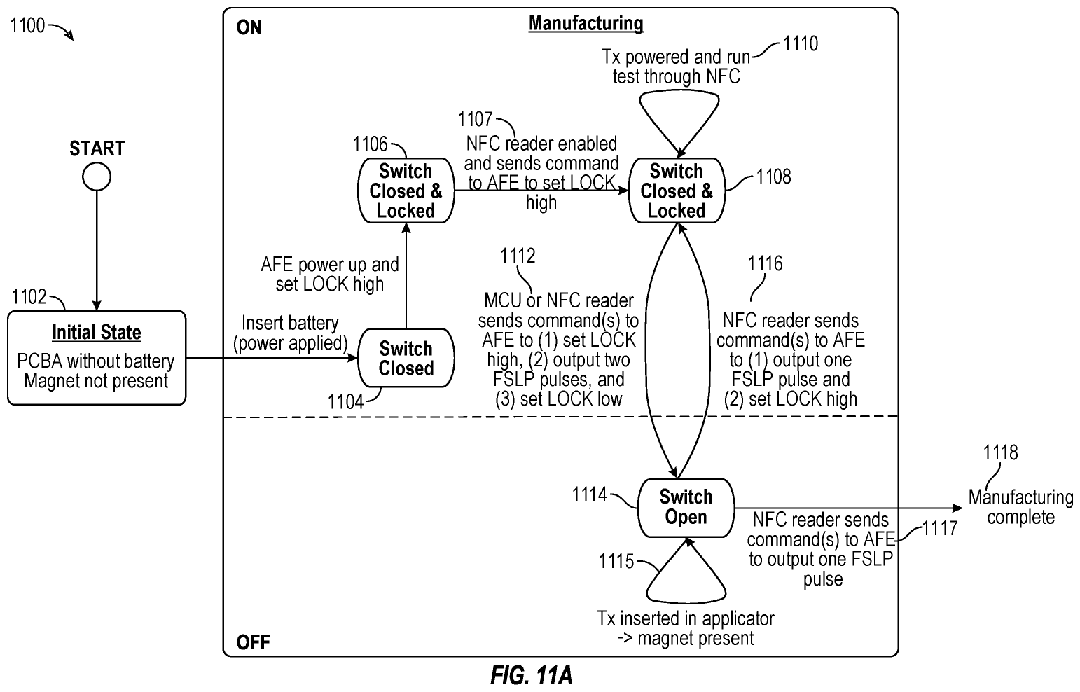

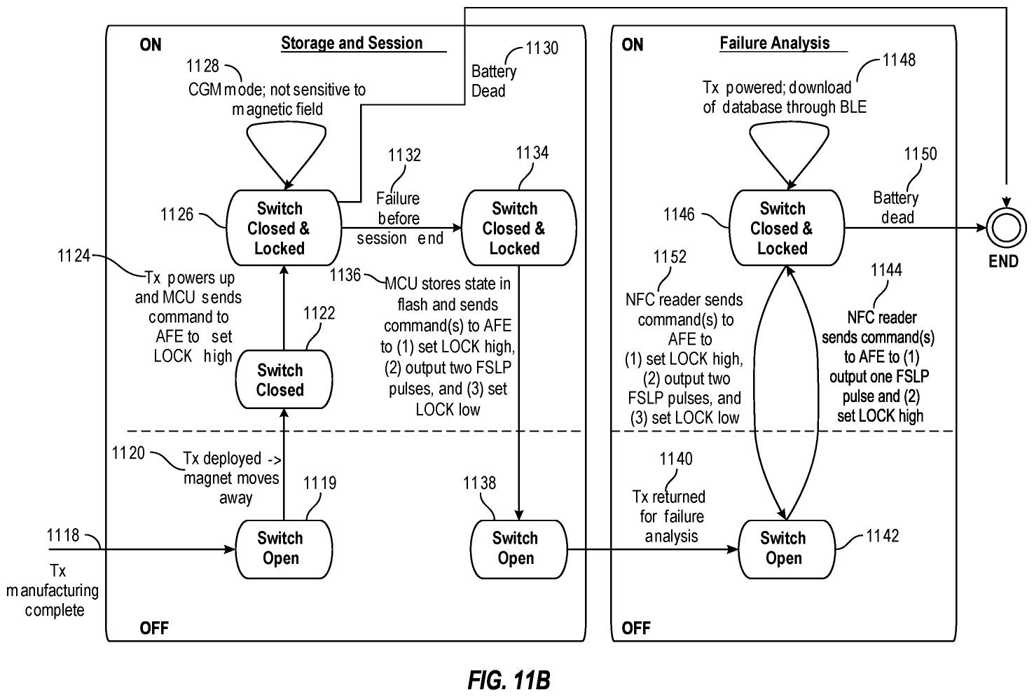

FIGS. 11A and 11B include a logic state diagram 1100 associated with the power switch 804 of the TMR sensor 802, illustrating the different power switch states/actions of Table 3. As shown in FIG. 11A, the logic state diagram 1100 begins in initial state 1102 in which the battery 806 of the analyte sensor system 800 illustrated in FIG. 10 is not inserted on to a PCBA of the analyte sensor system 800 and a magnetic field is not applied to the TMR sensor 802. Thereafter, during a manufacturing procedure of the analyte sensor system 800, the battery 806 may be inserted into the PCBA and the logic state diagram 1100 moves to state 1104 in which the power switch 804 is closed and the analyte sensor system 800 is powered on, corresponding to row 4 in Table 3. After powering on, the AFE component 808 outputs the LOCK signal 814 to the TMR sensor 802, locking the analyte sensor system 800 on, as shown in state 1106 corresponding to row 5 in Table 3.

Thereafter, the analyte sensor system 800 may be configured to perform one or more testing procedures to ensure proper operation of the analyte sensor system 800. For example, as shown at 1107, NFC reader 816 may send a command to the AFE component 808 to set the LOCK signal 814 high, resulting in the power switch 804 being closed and locked in state 1108 corresponding row 5 of Table 3. The analyte sensor system 800 may then perform one or more testing procedures based on one or more commands received from the NFC reader, as shown at 1110.

Thereafter, after the one or more testing procedures are complete, the MCU 810 or NFC reader 816 may instruct the AFE component 808 to open the power switch 804 to prevent the flow of current from the battery 806 and conserve power. To do this, as shown at 1112, the MCU 810 or NFC reader 816 may send one or more commands to the AFE component 808 to (1) set the LOCK signal 814 high on the LOCK pin 815 if not already set high, (2) output a first number of pulses of the FSLP signal 1004 (e.g., two pulses) on the FSLP pin 1002, and (3) after outputting the first number of pulses of the FSLP signal 1004, set the LOCK signal 814 low on the LOCK pin 815. While the first number of pulses of the FSLP signal 1004 is illustrated and described as being two pulses, it should be appreciated that the first number of pulses may be any positive whole number of pulses greater than zero.

The first number of pulses of the FSLP signal 1004 and the transition from the LOCK signal 814 being set high to the LOCK signal 814 being set low (e.g., regardless of whether a magnetic field is applied to the TMR sensor 802) may trigger the analyte sensor system 800 to enter a FSLP state in which the TMR sensor 802 is configured to open the power switch 804, as shown in state 1114 corresponding to the FSLP state shown in row 6 of Table 3. The power switch 804 may be maintained open in state 1114 (e.g., FSLP state) for long periods of time during the manufacturing process in order to conserve battery power. Only when the analyte sensor system 800 needs to be tested again is the analyte sensor system 800 transitioned out of the FSLP state and the power switch 804 closed, allowing the analyte sensor system 800 to be powered up again.

For example, as shown at 1116, in order to transition the analyte sensor system 800 out of the FSLP state, the NFC reader 816 may send a command to the AFE component 808 to output a second number of pulses of the FSLP signal 1004 (e.g., one pulse) on the FSLP pin 1002 to be received by the TMR sensor 802 on the FSLP pin 1006. While the second number of pulses of the FSLP signal 1004 is illustrated and described as being one pulse, it should be appreciated that the second number of pulses may be any positive whole number of pulses greater than zero and different from the first number of pulses.

Based on the one FSLP pulse received by the TMR sensor 802, the analyte sensor system 800 may transition out of the FSLP state in which the TMR sensor 802 is again under magnetic control, in accordance with row 7 of Table 3. Thereafter, the NFC reader 816 may send a command to the AFE component 808 to set the LOCK signal 814 high, resulting in the power switch 804 being closed and locked in state 1108 corresponding row 5 of Table 3. The analyte sensor system 800 may then perform one or more additional testing procedures based on one or more commands received from the NFC reader 816, as shown at 1110. Thereafter, as shown at 1112, after the one or more additional testing procedures are complete, the MCU 810 or NFC reader 816 again sends one or more commands to the AFE component 808 to (1) set the LOCK signal 814 high on the LOCK pin 815 if not already set high, (2) output the first number of pulses of the FSLP signal 1004 on the FSLP pin 1002, and (3) after outputting the first number of pulses of the FSLP signal 1004, set the LOCK signal 814 low on the LOCK pin 815. As noted above, the first number of pulses of the FSLP signal 1004 and the transition from the LOCK signal 814 being set high to the LOCK signal 814 being set low (e.g., regardless of whether a magnetic field is applied to the TMR sensor 802) may trigger the analyte sensor system 800 to enter a FSLP state in which the TMR sensor 802 is configured to open the power switch 804, as shown in state 1114 corresponding to the FSLP state shown in row 6 of Table 3.

At some point in time, as shown at 1115, the analyte sensor system 800 may be inserted into an applicator device (e.g., a device used to deploy the analyte sensor system 800 on to a user) that includes a magnet that applies a magnetic field to the TMR sensor 802. Because the FSLP state has been triggered, as described above, the power switch 804 remains open in state 1114 after the analyte sensor system 800 is inserted into the applicator device. In other words, state 1114 is maintained when the magnetic field is applied to the TMR sensor 802 in accordance with row 6 of Table 3.

As shown at 1117, after the analyte sensor system 800 has been inserted into the applicator device, the NFC reader 816 may send a command to the AFE component 808 to output the second number of pulses of the FSLP signal 1004 on the FSLP pin 1002 to be received by the TMR sensor 802 on the FSLP pin 1006. Based on the single FSLP pulse received by the TMR sensor 802, the analyte sensor system 800 may transition out of the FSLP state in which the TMR sensor 802 is again under magnetic control, in accordance with row 7 of Table 3. It should be appreciated that since the analyte sensor system 800 has been inserted into the applicator device that includes the magnet that applies the magnetic field to the TMR sensor 802, the power switch 804 may be maintained open, in accordance with row 2 of Table 3. Thereafter, as shown at 1118, the manufacturing procedure of the analyte sensor system 800 may be completed and the analyte sensor system 800 may be put into storage or shipped to user.

After the manufacturing process is completed, the logic state diagram 1100 then moves into a storage and session phase, as illustrated in FIG. 11B. For example, as shown in FIG. 11B, after the manufacturing procedure of the analyte sensor system is completed at 1118 and while in storage or being shipped to the user, the power switch 804 may be maintained in an open position, as shown in state 1119 corresponding to row 2 in Table 3. Thereafter, as shown at 1120, the analyte sensor system 800 may be deployed onto the user, resulting in the magnetic field no longer being applied to the TMR sensor 802 (e.g., since after being deployed, the magnet stays in the applicator while the analyte sensor system 800 is attached to the user). Due to the magnetic field no longer being applied to the TMR sensor 802, the TMR sensor 802 is configured to close the power switch 804, as shown in state 1122 corresponding to row 4 of Table 3.

Thereafter, current from the battery 806 may begin to flow, powering on the analyte sensor system 800. Further, as shown at 1124, the MCU 810 may send a command to the AFE component 808, instructing the AFE component 808 to set the LOCK signal 814 high (e.g., instructing the AFE component 808 to output a high LOCK signal 814). Based on the LOCK signal 814, the power switch 804 may be maintained/locked in a closed position, as shown in state 126 corresponding to row 5 in Table 3. While the power switch 804 is closed and locked, the analyte sensor system 800 may operate in a CGM mode and may not be sensitive to changes in a magnetic field, as shown at 1128. While operating in the CGM mode, the analyte sensor system 800 may perform one or more analyte measurements of the user and transmit these analyte measurements to a display device (e.g., display devices 110, 120, 130, and/or 140).

The analyte sensor system 800 may operate in the CGM mode until either the battery 806 is depleted, as shown at 1130, or a failure of the analyte sensor system 800 occurs, as shown at 1132. When a failure of the analyte sensor system 800 occurs, the power switch 804 may still be maintained in a closed position and locked, as shown in state 1134, which may cause the analyte sensor system 800 to continue to consume power from the battery 806. To ensure enough power remains to perform diagnostics on the failure at a later time, when the failure is detected, the MCU 810 may store within the non-volatile memory of the analyte sensor system 800 (e.g., storage 515 illustrated in FIG. 5) diagnostic information indicating the state of the analyte sensor system 800 as shown at 1136. As noted above, diagnostic information indicating the state of the analyte sensor system 800 may include information such as time of failure, type of failure, MCU register values, random access memory (RAM) data.

Thereafter, as shown at 1136, to put the analyte sensor system 800 into the FSLP state to conserve battery power, the MCU 810 may instruct the AFE component 808 to output the first number of pulses of the FSLP signal 1004 on the FSLP pin 1002 and, after outputting the first number of pulses of the FSLP signal 1004, set the LOCK signal 814 low on the LOCK pin 815, in accordance with row 6 of Table 3. Based on the first number of pulses of the FSLP signal 1004 and the transition of the LOCK signal 814 being set high to the LOCK signal 814 being set low, the TMR sensor 802 may be configured to open the power switch 804, powering off the analyte sensor system 800, as shown in state 1138 corresponding to row 6 of Table 2.

After powering down, the analyte sensor system 800 may be sent back to the manufacturer or retailer for failure analysis as shown at 1140. As shown, during shipment, the power switch 804 may be maintained in an open position, as shown in state 1142 corresponding to the FSLP state illustrated in row 6 of Table 3. Once received, as shown at 1144, the manufacturer or retailer may use an NFC reader 816 to transmit an energy signal to power (e.g., using energy harvesting circuitry) and instruct the AFE component 808 to output the second number of pulses of the FSLP signal 1004 to the TMR sensor 802, causing the analyte sensor system 800 to exit the FSLP state and the TMR sensor 802 to return to magnetic control, as shown in row 7 of Table 3. In some embodiments, the NFC reader 816 may also instruct the AFE component 808 to set the LOCK signal 814 high.

In response, the TMR sensor 802 may be configured to close the power switch 804 and lock it in the closed position, as shown in state 1146 corresponding to row 5 of Table 3, allowing the analyte sensor system 800 to power on again using any remaining power stored in the battery 806. While the analyte sensor system 800 is powered on, the manufacturer or retailer may download the diagnostic information indicating the state of the analyte sensor system 800 from the memory of the analyte sensor system 800, as shown at 1148. The manufacturer or retailer may then perform diagnostics using the diagnostic information to determine a cause of the failure the analyte sensor system 800. In some embodiments, the manufacturer or retailer may download the diagnostic information using BLE, WiFi, cellular based wireless communications, a hardwire connection, etc.

In some cases, the power switch 804 may be maintained in the closed position in state 1146 until the battery 806 is depleted as shown at 1150. In other cases, as shown at 1152, the manufacturer or retailer may decide to conserve power of the battery 806 during failure analysis and use the NFC reader 816 to instruct the AFE component 808 to (1) set the LOCK signal 814 high on the LOCK pin 815 if not already set high, (2) output the first number of pulses of the FSLP signal 1004 on the FSLP pin 1002, and (3) after outputting the first number of pulses of the FSLP signal 1004, set the LOCK signal 814 low on the LOCK pin 815. As a result, the analyte sensor system 800 may enter the FSLP state, causing the TMR sensor 802 to open the power switch 804 and return to state 1142 in accordance with row 6 of Table 3.

### Example Operations of an Analyte Sensor System

FIG. 12 shows an example of a method 1200 for operating a power switch at of analyte sensor system, such the analyte sensor system 208 of FIG. 5 and/or the analyte sensor system 800 of FIGS. 8 and/or 10.

Method 1200 begins at 1205 with detecting, by a power control sensor (e.g., TMR sensor 802) of the analyte sensor system, whether a signal is applied to the power control sensor.

Method 1200 continues at 1210 with receiving, at an analog front end (AFE) component (e.g., AFE component 808) of the analyte sensor system from at least one of a near field communication (NFC) reader device (e.g., NFC reader device 816) or one or more other electrical components (e.g., MCU 810) of the analyte sensor system, a second set of control signals for instructing the power control sensor to operate a power switch (e.g., power switch 804) to control current flow from a battery (e.g., battery 806) of the analyte sensor system to at least the transcutaneous analyte sensor, the AFE component, and the one or more other electrical components.

Method 1200 continues at 1215 with outputting, by the AFE component based on the second set of control signals, a first set of control signals for instructing the power control sensor to operate the power switch.

Method 1200 continues at 1220 with receiving, by the power control sensor, the first set of control signals from the AFE component.

Method 1200 continues at 1225 with operating, by the power control sensor based on the first set of control signals and the detection of whether the signal is applied to the power control sensor, the power switch to control the current flow from the battery of the analyte sensor system to at least the transcutaneous analyte sensor, the AFE component, and the one or more other electrical components.

In some embodiments, method 1200 further includes harvesting, by the AFE component, energy from the second set of control signals for powering the AFE component, wherein outputting the first set of control signals is based on the energy harvested from the second set of control signals.

In some embodiments, the power control sensor comprises a tunnel magnetoresistance (TMR) sensor. In some embodiments, the signal applied to the power control sensor comprises a magnetic field.

In some embodiments, receiving the second set of control signals comprises receiving the second set of control signals from the NFC reader during a manufacturing procedure of the analyte sensor system or after the manufacturing procedure has been completed. In some embodiments, the second set of control signals instruct the AFE component to output the first set of control signals to the power control sensor to open the power switch. In some embodiments, based on the first set of control signals, operating the power switch at 1225 comprises opening the power switch to prevent the current flow from the battery to at least the transcutaneous analyte sensor, the AFE component, and the one or more other electrical components.

In some embodiments, method 1200 further includes receiving, by the AFE component during the manufacturing procedure, a third set of control signals from the NFC reader device instructing the AFE component to output a fourth set of control signals to the power control sensor to close the power switch. In some embodiments, based on the fourth set of control signals from the AFE component, operating the power switch at 1225 comprises closing the power switch to allow the current flow from the battery to at least the transcutaneous analyte sensor, the AFE component, and the one or more other electrical components.

In some embodiments, method 1200 further includes performing, by the one or more other electrical components, one or more testing procedures associated with the analyte sensor system based on the third set of control signals received from the NFC reader device and the current flow from the battery.

In some embodiments, method 1200 further includes detecting, by the one or more other electrical components, a failure of the analyte sensor system. In some embodiments, method 1200 further includes outputting, by the one or more other electrical components based on the detected failure, a third set of control signals instructing AFE component to output a fourth set of control signals to the power control sensor to open the power switch. In some embodiments, based on the fourth set of control signals, operating the power switch at 1225 comprises opening the power switch to prevent the current flow from the battery to at least the transcutaneous analyte sensor, the AFE component, and the one or more other electrical components.

In some embodiments, receiving the first set of control signals from the AFE component at 1220 comprises using a set of control pins (e.g., LOCK pin 817 and/or FSLP pin 1006) of the power control sensor to receive the first set of control signals from the AFE component.

In some embodiments, the first set of control signals comprises a LOCK signal and a force sleep (FSLP) signal. In some embodiments, the set of control pins comprises two control pins including a LOCK pin for receiving the LOCK signal and a FSLP pin for receiving the FSLP signal.

In some embodiments, method 1200 further comprises setting, by the AFE component, the LOCK signal high based on the second set of control signals. In some embodiments, operating the power switch at 1225 comprises closing the power switch to allow the current flow from the battery based on the LOCK signal being set high.

In some embodiments, closing the power switch based on the LOCK signal being set high comprises closing the power switch based on the LOCK signal being set high regardless of whether or not the signal is applied to the power control sensor.

In some embodiments, method 1200 further comprises, based on the second set of control signals: outputting, by the AFE component, a first number of pulses of the FSLP signal and setting, by the AFE component, the LOCK signal low after outputting the first number of pulses of the FSLP signal. In some embodiments, based on the first number of pulses of the FSLP signal and the LOCK signal being set low, operating the power switch at 1225 comprises opening the power switch to prevent the current flow from the battery and to cause the analyte sensor system to enter a FSLP state.

In some embodiments, regardless of whether or not the signal is applied to the power control sensor while the analyte sensor system is in the FSLP state, operating the power switch at 1225 comprises maintaining the power switch open to prevent the current flow from the battery.

In some embodiments, method 1200 further comprises, while the analyte sensor is in the FSLP state: setting, by the AFE component, the LOCK signal low based on the second set of control signals and outputting, by the AFE component, a second number of pulses of the FSLP signal.

In some embodiments, method 1200 further includes exiting, by the analyte sensor system, the FSLP state based on the LOCK signal being set low and the second number of pulses of the FSLP signal.

In some embodiments, the first set of control signals comprises a LOCK signal. In some embodiments, the set of control pins comprises one control pin including a LOCK pin for receiving the LOCK signal.

In some embodiments, method 1200 further comprises setting, by the AFE component, the LOCK signal high based on the second set of control signals. In some embodiments, operating the power switch at 1225 comprises closing the power switch to allow the current flow from the battery based on the LOCK signal being set high.

In some embodiments, closing the power switch comprises closing the power switch based on the LOCK signal being set high regardless of whether or not the signal is applied to the power control sensor.

In some embodiments, method 1200 further comprises setting, by the AFE component, the LOCK signal low based on the second set of control signals. In some embodiments, based on a transition of the LOCK signal being set high to the LOCK signal being set low, operating the power switch at 1225 comprises opening the power switch to prevent the current flow from the battery.

In some embodiments, detecting whether the signal is applied to the power control sensor at 1205 comprises detecting a transition of the signal being applied to the power control sensor to the signal not being applied to the power control sensor. In some embodiments, based on the detected transition, operating the power switch at 1225 comprises closing the power switch to allow the current flow from the battery regardless of the LOCK signal.

### Example Clauses

Implementation examples are described in the following numbered clauses:

Clause 1: An analyte sensor system, comprising: a power switch; a power control sensor; an analog front end (AFE) component; and one or more other electrical components, wherein: the power control sensor is configured to detect whether a signal is applied to the power control sensor and, based on the detection, operate the power switch to control current flow from a battery of the analyte sensor system to at least a transcutaneous analyte sensor of the analyte sensor system, the AFE component, and the one or more other electrical components; the power control sensor is configured to receive a first set of control signals from the AFE component for instructing the power control sensor to operate the power switch; and the AFE is configured to receive a second set of control signals from at least one of a near field communication (NFC) reader device or the one or more other electrical components instructing the AFE to output the first set of control signals for instructing the power control sensor to operate the power switch.

Clause 2: The analyte sensor system of Clause 1, wherein: the AFE component further includes circuitry for harvesting energy from the second set of control signals for powering the AFE; and the AFE component is configured to output the first set of control signals to the power control sensor based on the energy harvested from the second set of control signals.

Clause 3: The analyte sensor system of any of Clauses 1-2, wherein: the power control sensor comprises a tunnel magnetoresistance (TMR) sensor; and the signal applied to the power control sensor comprises a magnetic field.

Clause 4: The analyte sensor system of any of Clauses 1-3, wherein: the AFE component is configured to receive the second set of control signals from the NFC reader during a manufacturing procedure of the analyte sensor system or after the manufacturing procedure has been completed; and the second set of control signals instruct the AFE component to output the first set of control signals to the power control sensor to open the power switch; and based on the first set of control signals, the power control sensor is configured to open the power switch to prevent the current flow from the battery to at least the transcutaneous analyte sensor, the AFE component, and the one or more other electrical components.

Clause 5: The analyte sensor system of Clause 4, wherein: the AFE component is configured to receive, during the manufacturing procedure, a third set of control signals from the NFC reader device instructing the AFE component to output a fourth set of control signals to the power control sensor to close the power switch; based on the fourth set of control signals from the AFE component, the power control sensor is configured to close the power switch to allow the current flow from the battery to at least the transcutaneous analyte sensor, the AFE component, and the one or more other electrical components; and the one or more other electrical components are configured to perform one or more testing procedures associated with the analyte sensor system based on the third set of control signals received from the NFC reader device and the current flow from the battery.

Clause 6: The analyte sensor system of any of Clauses 1-5, wherein the one or more other electrical components are configured to: detect a failure of the analyte sensor system; and output, based on the detected failure, a third set of control signals instructing the power control sensor to open the power switch to prevent the current flow from the battery to at least the transcutaneous analyte sensor, the AFE component, and the one or more other electrical components.

Clause 7: The analyte sensor system of any of Clauses 1-6, wherein the power control sensor includes a set of control pins for receiving the first set of control signals from the AFE component.

Clause 8: The analyte sensor system of Clause 7, wherein: the first set of control signals comprises a LOCK signal and a force sleep (FSLP) signal; and the set of control pins comprises two control pins including a LOCK pin for receiving the LOCK signal and a FSLP pin for receiving the FSLP signal.

Clause 9: The analyte sensor system of Clause 8, wherein: based on the second set of control signals, the AFE component is configured to set the LOCK signal high; and the power control sensor is configured to close the power switch to allow the current flow from the battery based on the LOCK signal being set high.

Clause 10: The analyte sensor system of Clause 9, wherein the power control sensor is configured to close the power switch based on the LOCK signal being set high regardless of whether or not the signal is applied to the power control sensor.

Clause 11: The analyte sensor system of any of Clauses 9-10, wherein: based on the second set of control signals, the AFE component is configured to: output a first number of pulses of the FSLP signal; and set the LOCK signal low after outputting the first number of pulses of the FSLP signal; and based on the first number of pulses of the FSLP signal and the LOCK signal being set low, the power control sensor is configured to open the power switch to prevent the current flow from the battery and to cause the analyte sensor system to enter a FSLP state.

Clause 12: The analyte sensor system of Clause 11, wherein, regardless of whether or not the signal is applied to the power control sensor while the analyte sensor system is in the FSLP state, the power control sensor is configured to maintain the power switch open to prevent the current flow from the battery.

Clause 13: The analyte sensor system of Clause 12, wherein, while the analyte sensor is in the FSLP state, the AFE component is configured to: set the LOCK signal low based on the second set of control signals; and output a second number of pulses of the FSLP signal.

Clause 14: The analyte sensor system of Clause 13, wherein the analyte sensor system is configured to exit the FSLP state based on the LOCK signal being set low and the second number of pulses of the FSLP signal.

Clause 15: The analyte sensor system of Clause 7, wherein: the first set of control signals comprises a LOCK signal; and the set of control pins comprises one control pin including a LOCK pin for receiving the LOCK signal.

Clause 16: The analyte sensor system of Clause 15, wherein: based on the second set of control signals, the AFE component is configured to set the LOCK signal high; and the power control sensor is configured to close the power switch to allow the current flow from the battery based on the LOCK signal being set high.

Clause 17: The analyte sensor system of Clause 16, wherein the power control sensor is configured to close the power switch based on the LOCK signal being set high regardless of whether or not the signal is applied to the power control sensor.

Clause 18: The analyte sensor system of any of Clauses 16-17, wherein: based on the second set of control signals, the AFE component is configured to set the LOCK signal low; and based on a transition of the LOCK signal being set high to the LOCK signal being set low, the power control sensor is configured to open the power switch to prevent the current flow from the battery.

Clause 19: The analyte sensor system of Clause 18, wherein: the power control sensor is configured to detect a transition of the signal being applied to the power control sensor to the signal not being applied to the power control sensor; and based on the detected transition, the power control sensor is configured to close the power switch to allow the current flow from the battery regardless of the LOCK signal.

Clause 20: The analyte sensor system of any of Clauses 1-19, wherein: the transcutaneous analyte sensor is configured to measure one or more analyte levels of a user of the analyte sensor system; the battery is configured to supply current to at least the transcutaneous analyte sensor, the AFE component, and the one or more other electrical components; and the power switch is configured provide a path for current flow from the battery to at least the transcutaneous analyte sensor, the AFE component, and the one or more other electrical components.

Clause 21: A method for operating a power switch at an analyte sensor system, comprising: detecting, by a power control sensor of the analyte sensor system, whether a signal is applied to the power control sensor; receiving, by an analog front end (AFE) component of the analyte sensor system from at least one of a near field communication (NFC) reader device or one or more other electrical components of the analyte sensor system, a second set of control signals for instructing the power control sensor to operate a power switch to control current flow from a battery of the analyte sensor system to at least the transcutaneous analyte sensor, the AFE component, and the one or more other electrical components; outputting, by the AFE component based on the second set of control signals, a first set of control signals for instructing the power control sensor to operate the power switch; receiving, by the power control sensor, the first set of control signals from the AFE component; and operating, by the power control sensor based on the first set of control signals and the detection of whether the signal is applied to the power control sensor, the power switch to control the current flow from the battery of the analyte sensor system to at least the transcutaneous analyte sensor, the AFE component, and the one or more other electrical components.

Clause 22: The method of Clause 21, further comprising harvesting, by the AFE component, energy from the second set of control signals for powering the AFE component, wherein outputting the first set of control signals is based on the energy harvested from the second set of control signals.

Clause 23: The method of any of Clauses 21-22, wherein: the power control sensor comprises a tunnel magnetoresistance (TMR) sensor; and the signal applied to the power control sensor comprises a magnetic field.

Clause 24: The method of any of Clauses 21-23, wherein: receiving the second set of control signals comprises receiving the second set of control signals from the NFC reader during a manufacturing procedure of the analyte sensor system or after the manufacturing procedure has been completed; the second set of control signals instruct the AFE component to output the first set of control signals to the power control sensor to open the power switch; and based on the first set of control signals, operating the power switch comprises opening the power switch to prevent the current flow from the battery to at least the transcutaneous analyte sensor, the AFE component, and the one or more other electrical components.

Clause 25: The method of Clause 24, further comprising receiving, by the AFE component during the manufacturing procedure, a third set of control signals from the NFC reader device instructing the AFE component to output a fourth set of control signals to the power control sensor to close the power switch, wherein: based on the fourth set of control signals from the AFE component, operating the power switch comprises closing the power switch to allow the current flow from the battery to at least the transcutaneous analyte sensor, the AFE component, and the one or more other electrical components.

Clause 26: The method of Clause 25, further comprising performing, by the one or more other electrical components, one or more testing procedures associated with the analyte sensor system based on the third set of control signals received from the NFC reader device and the current flow from the battery.

Clause 27: The method of any of Clauses 21-26, further comprising: detecting, by the one or more other electrical components, a failure of the analyte sensor system; and outputting, by the one or more other electrical components based on the detected failure, a third set of control signals instructing AFE component to output a fourth set of control signals to the power control sensor to open the power switch; and based on the fourth set of control signals, operating the power switch comprises opening the power switch to prevent the current flow from the battery to at least the transcutaneous analyte sensor, the AFE component, and the one or more other electrical components.

Clause 28: The method of any of Clauses 21-27, wherein receiving the first set of control signals from the AFE component comprises using a set of control pins of the power control sensor to receive the first set of control signals from the AFE component.

Clause 29: The method of Clause 28, wherein: the first set of control signals comprises a LOCK signal and a force sleep (FSLP) signal; and the set of control pins comprises two control pins including a LOCK pin for receiving the LOCK signal and a FSLP pin for receiving the FSLP signal.

Clause 30: The method of Clause 29, further comprising setting, by the AFE component, the LOCK signal high based on the second set of control signals, wherein operating the power switch comprises closing the power switch to allow the current flow from the battery based on the LOCK signal being set high.

Clause 31: The method of Clause 30, wherein closing the power switch based on the LOCK signal being set high comprises closing the power switch based on the LOCK signal being set high regardless of whether or not the signal is applied to the power control sensor.

Clause 32: The method of any of Clauses 30-31, further comprising, based on the second set of control signals: outputting, by the AFE component, a first number of pulses of the FSLP signal; and setting, by the AFE component, the LOCK signal low after outputting the first number of pulses of the FSLP signal, wherein: based on the first number of pulses of the FSLP signal and the LOCK signal being set low, operating the power switch comprises opening the power switch to prevent the current flow from the battery and to cause the analyte sensor system to enter a FSLP state.

Clause 33: The method of Clause 32, wherein, regardless of whether or not the signal is applied to the power control sensor while the analyte sensor system is in the FSLP state, operating the power switch comprises maintaining the power switch open to prevent the current flow from the battery.

Clause 34: The method of Clause 33, further comprising, while the analyte sensor is in the FSLP state: setting, by the AFE component, the LOCK signal low based on the second set of control signals; and outputting, by the AFE component, a second number of pulses of the FSLP signal.

Clause 35: The method of Clause 34, further comprising exiting, by the analyte sensor system, the FSLP state based on the LOCK signal being set low and the second number of pulses of the FSLP signal.

Clause 36: The method of Clause 28, wherein: the first set of control signals comprises a LOCK signal; and the set of control pins comprises one control pin including a LOCK pin for receiving the LOCK signal.

Clause 37: The method of Clause 36, further comprising setting, by the AFE component, the LOCK signal high based on the second set of control signals, wherein operating the power switch comprises closing the power switch to allow the current flow from the battery based on the LOCK signal being set high.

Clause 38: The method of Clause 37, wherein closing the power switch comprises closing the power switch based on the LOCK signal being set high regardless of whether or not the signal is applied to the power control sensor.

Clause 39: The method of any of Clauses 37-38, further comprising setting, by the AFE component, the LOCK signal low based on the second set of control signals, wherein based on a transition of the LOCK signal being set high to the LOCK signal being set low, operating the power switch comprises opening the power switch to prevent the current flow from the battery.

Clause 40: The method of Clause 39, wherein: detecting whether the signal is applied to the power control sensor comprises detecting a transition of the signal being applied to the power control sensor to the signal not being applied to the power control sensor; and based on the detected transition, operating the power switch comprises closing the power switch to allow the current flow from the battery regardless of the LOCK signal.

Clause 41: An analyte monitoring system, comprising: an analyte sensor system configured to perform analyte measurements associated with a user of the analyte sensor system; and a display device configured to receive the analyte measurements from the analyte sensor system, wherein: the analyte sensor system includes: a power switch; a power control sensor; an analog front end (AFE) component; and one or more other electrical components; the power control sensor is configured to detect whether a signal is applied to the power control sensor and, based on the detection, operate the power switch to control current flow from a battery of the analyte sensor system to at least a transcutaneous analyte sensor of the analyte sensor system, the AFE component, and the one or more other electrical components; the power control sensor is configured to receive a first set of control signals from the AFE component for instructing the power control sensor to operate the power switch; and the AFE is configured to receive a second set of control signals from at least one of a near field communication (NFC) reader device or the one or more other electrical components instructing the AFE to output the first set of control signals for instructing the power control sensor to operate the power switch.

Clause 42: The analyte monitoring system of Clause 41, wherein the power control sensor includes a set of control pins for receiving the first set of control signals from the AFE component.

Clause 43: The analyte monitoring system of Clause 42, wherein: the first set of control signals comprises a LOCK signal and a force sleep (FSLP) signal; and the set of control pins comprises two control pins including a LOCK pin for receiving the LOCK signal and a FSLP pin for receiving the FSLP signal.

Clause 44: The analyte monitoring system of Clause 43, wherein: based on the second set of control signals, the AFE component is configured to set the LOCK signal high; and the power control sensor is configured to close the power switch to allow the current flow from the battery based on the LOCK signal being set high.

Clause 45: The analyte monitoring system of Clause 44, wherein the power control sensor is configured to close the power switch based on the LOCK signal being set high regardless of whether or not the signal is applied to the power control sensor.

Clause 46: The analyte monitoring system of any of Clauses 44-45, wherein: based on the second set of control signals, the AFE component is configured to: output a first number of pulses of the FSLP signal; and set the LOCK signal low after outputting the first number of pulses of the FSLP signal; and based on the first number of pulses of the FSLP signal and the LOCK signal being set low, the power control sensor is configured to open the power switch to prevent the current flow from the battery and to cause the analyte sensor system to enter a FSLP state.

Clause 47: The analyte monitoring system of Clause 46, wherein, regardless of whether or not the signal is applied to the power control sensor while the analyte sensor system is in the FSLP state, the power control sensor is configured to maintain the power switch open to prevent the current flow from the battery.

Clause 48: The analyte monitoring system of Clause 47, wherein, while the analyte sensor system is in the FSLP state, the AFE component is configured to: set the LOCK signal low based on the second set of control signals; and output a second number of pulses of the FSLP signal.

Clause 49: An analyte sensor system, comprising: a transcutaneous analyte sensor; a battery; a power switch; a power control sensor; an analog front end (AFE) component; and one or more other electrical components, wherein: the transcutaneous analyte sensor is configured to measure one or more analyte levels of a user of the analyte sensor system; the battery is configured to supply current to at least the transcutaneous analyte sensor, the AFE component, and the one or more other electrical components; the power switch is configured provide a path for current flow from the battery to at least the transcutaneous analyte sensor, the AFE component, and the one or more other electrical components; the power control sensor is configured to detect whether a signal is applied to the power control sensor and, based on the detection, operate the power switch to control the current flow from the battery to at least the transcutaneous analyte sensor, the AFE component, and the one or more other electrical components; the power control sensor includes a set of control pins configured to receive a first set of control signals from the AFE component for instructing the power control sensor to operate the power switch; and the AFE is configured to receive a second set of control signals from at least one of a near field communication (NFC) reader device or the one or more other electrical components instructing the AFE to output the first set of control signals for instructing the power control sensor to operate the power switch.

### Additional Considerations

In this document, the terms “computer program medium” and “computer usable medium” and “computer readable medium”, as well as variations thereof, are used to generally refer to transitory or non-transitory media. These and other various forms of computer program media or computer usable/readable media may be involved in carrying one or more sequences of one or more instructions to a processing device for execution. Such instructions embodied on the medium, may generally be referred to as “computer program code” or a “computer program product” or “instructions” (which may be grouped in the form of computer programs or other groupings). When executed, such instructions may enable a computing module, such as the analyte sensor system 208 and/or analyte sensor system 800, circuitry related thereto, and/or a processor thereof or connected thereto to perform features or functions of the present disclosure as discussed herein (for example, in connection with methods described above and/or in the claims), including for example when the same is/are incorporated into a system, apparatus, device and/or the like.

Various embodiments have been described with reference to specific example features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense. It will be appreciated that, for clarity purposes, the above description has described embodiments with reference to different functional units. However, it will be apparent that any suitable distribution of functionality between different functional units may be used without detracting from the invention. For example, functionality illustrated to be performed by separate computing devices may be performed by the same computing device. Likewise, functionality illustrated to be performed by a single computing device may be distributed amongst several computing devices. Hence, references to specific functional units are only to be seen as references to suitable means for providing the described functionality, rather than indicative of a strict logical or physical structure or organization.

Although described above in terms of various example embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the present application, whether or not such embodiments are described and whether or not such features are presented as being a part of a described embodiment. Thus, the breadth and scope of the present application should not be limited by any of the above-described example embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide illustrative instances of the item in discussion, not an exhaustive or limiting list thereof; the terms “a” or “an” should be read as meaning “at least one,” “one or more” or the like; the term “set” should be read to include one or more objects of the type included in the set; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Similarly, the plural may in some cases be recognized as applicable to the singular and vice versa. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic, circuitry, or other components, may be combined in a single package or separately maintained and may further be distributed in multiple groupings or packages or across multiple locations.

Additionally, the various embodiments set forth herein are described in terms of example block diagrams, flow charts, and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration. Moreover, the operations and sub-operations of various methods described herein are not necessarily limited to the order described or shown in the figures, and one of skill in the art will appreciate, upon studying the present disclosure, variations of the order of the operations described herein that are within the spirit and scope of the disclosure. It will be understood that each block of the flowchart illustrations, and combinations of blocks in the flowchart illustrations, can be implemented by execution of computer program instructions. These computer program instructions may be loaded onto a computer or other programmable data processing apparatus (such as a controller, microcontroller, microprocessor or the like) in a sensor electronics system to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks presented herein.

It should be appreciated that all methods and processes disclosed herein may be used in any glucose or other analyte monitoring system, continuous or intermittent. It should further be appreciated that the implementation and/or execution of all methods and processes may be performed by any suitable devices or systems, whether local or remote. Further, any combination of devices or systems may be used to implement the present methods and processes.

In addition, the operations and sub-operations of methods described herein may be carried out or implemented, in some cases, by one or more of the components, elements, devices, modules, circuitry, processors, etc. of systems, apparatuses, devices, environments, and/or computing modules described herein and referenced in various of figures of the present disclosure, as well as one or more sub-components, elements, devices, modules, processors, circuitry, and the like depicted therein and/or described with respect thereto. In such instances, the description of the methods or aspects thereof may refer to a corresponding component, element, etc., but regardless of whether an explicit reference is made, one of skill in the art will recognize upon studying the present disclosure when the corresponding component, element, etc. may be used. Further, it will be appreciated that such references do not necessarily limit the described methods to the particular component, element, etc. referred to. Thus, it will be appreciated by one of skill in the art that aspects and features described above in connection with (sub-) components, elements, devices, modules, and circuitry, etc., including variations thereof, may be applied to the various operations described in connection with methods described herein, and vice versa, without departing from the scope of the present disclosure.

What is claimed is:

1. An analyte sensor system, comprising:
a power switch;
a power control sensor;
an analog front end (AFE) component; and
one or more other electrical components, wherein:
the power control sensor is configured to detect whether a signal is applied to the power control sensor and, based on the detection, operate the power switch to control current flow from a battery of the analyte sensor system to at least a transcutaneous analyte sensor of the analyte sensor system, the AFE component, and the one or more other electrical components;
the power control sensor is configured to receive a first set of control signals from the AFE component for instructing the power control sensor to operate the power switch; and
the AFE component is configured to receive a second set of control signals from at least one of a near field communication (NFC) reader device or the one or more other electrical components instructing the AFE component to output the first set of control signals for instructing the power control sensor to operate the power switch.

2. The analyte sensor system of claim 1, wherein:
the AFE component further includes circuitry for harvesting energy from the second set of control signals for powering the AFE component; and
the AFE component is configured to output the first set of control signals to the power control sensor based on the energy harvested from the second set of control signals.

3. The analyte sensor system of claim 1, wherein:
the power control sensor comprises a tunnel magnetoresistance (TMR) sensor; and
the signal applied to the power control sensor comprises a magnetic field.

4. The analyte sensor system of claim 1, wherein:
the AFE component is configured to receive the second set of control signals from the NFC reader during a manufacturing procedure of the analyte sensor system or after the manufacturing procedure has been completed; and
the second set of control signals instruct the AFE component to output the first set of control signals to the power control sensor to open the power switch; and
based on the first set of control signals, the power control sensor is configured to open the power switch to prevent the current flow from the battery to at least the transcutaneous analyte sensor, the AFE component, and the one or more other electrical components.

5. The analyte sensor system of claim 4, wherein:
the AFE component is configured to receive, during the manufacturing procedure, a third set of control signals from the NFC reader device instructing the AFE component to output a fourth set of control signals to the power control sensor to close the power switch;
based on the fourth set of control signals from the AFE component, the power control sensor is configured to close the power switch to allow the current flow from the battery to at least the transcutaneous analyte sensor, the AFE component, and the one or more other electrical components; and
the one or more other electrical components are configured to perform one or more testing procedures associated with the analyte sensor system based on the third set of control signals received from the NFC reader device and the current flow from the battery.

6. The analyte sensor system of claim 1, wherein the one or more other electrical components are configured to:
detect a failure of the analyte sensor system; and
output, based on the detected failure, a third set of control signals instructing the power control sensor to open the power switch to prevent the current flow from the battery to at least the transcutaneous analyte sensor, the AFE component, and the one or more other electrical components.

7. The analyte sensor system of claim 1, wherein the power control sensor includes a set of control pins for receiving the first set of control signals from the AFE component.

8. The analyte sensor system of claim 7, wherein:
the first set of control signals comprises a LOCK signal and a force sleep (FSLP) signal; and
the set of control pins comprises two control pins including a LOCK pin for receiving the LOCK signal and a FSLP pin for receiving the FSLP signal.

9. The analyte sensor system of claim 8, wherein:
based on the second set of control signals, the AFE component is configured to set the LOCK signal high; and
the power control sensor is configured to close the power switch to allow the current flow from the battery based on the LOCK signal being set high.

10. The analyte sensor system of claim 9, wherein the power control sensor is configured to close the power switch based on the LOCK signal being set high regardless of whether or not the signal is applied to the power control sensor.

11. The analyte sensor system of claim 9, wherein:
based on the second set of control signals, the AFE component is configured to:
output a first number of pulses of the FSLP signal; and
set the LOCK signal low after outputting the first number of pulses of the FSLP signal; and
based on the first number of pulses of the FSLP signal and the LOCK signal being set low, the power control sensor is configured to open the power switch to prevent the current flow from the battery and to cause the analyte sensor system to enter a FSLP state.

12. The analyte sensor system of claim 11, wherein, regardless of whether or not the signal is applied to the power control sensor while the analyte sensor system is in the FSLP state, the power control sensor is configured to maintain the power switch open to prevent the current flow from the battery.

13. The analyte sensor system of claim 12, wherein, while the analyte sensor system is in the FSLP state, the AFE component is configured to:
set the LOCK signal low based on the second set of control signals; and
output a second number of pulses of the FSLP signal.

14. The analyte sensor system of claim 13, wherein the analyte sensor system is configured to exit the FSLP state based on the LOCK signal being set low and the second number of pulses of the FSLP signal.

15. A method for operating a power switch at an analyte sensor system, comprising:
detecting, by a power control sensor of the analyte sensor system, whether a signal is applied to the power control sensor;
receiving, by an analog front end (AFE) component of the analyte sensor system from at least one of a near field communication (NFC) reader device or one or more other electrical components of the analyte sensor system, a second set of control signals for instructing the power control sensor to operate a power switch to control current flow from a battery of the analyte sensor system to at least a transcutaneous analyte sensor of the analyte sensor system, the AFE component, and the one or more other electrical components;
outputting, by the AFE component based on the second set of control signals, a first set of control signals for instructing the power control sensor to operate the power switch;
receiving, by the power control sensor, the first set of control signals from the AFE component; and
operating, by the power control sensor based on the first set of control signals and the detection of whether the signal is applied to the power control sensor, the power switch to control the current flow from the battery of the analyte sensor system to at least the transcutaneous analyte sensor, the AFE component, and the one or more other electrical components.

16. The method of claim 15, wherein:
receiving the second set of control signals comprises receiving the second set of control signals from the NFC reader during a manufacturing procedure of the analyte sensor system or after the manufacturing procedure has been completed;
the second set of control signals instruct the AFE component to output the first set of control signals to the power control sensor to open the power switch; and
based on the first set of control signals, operating the power switch comprises opening the power switch to prevent the current flow from the battery to at least the transcutaneous analyte sensor, the AFE component, and the one or more other electrical components.

17. The method of claim 16, further comprising receiving, by the AFE component during the manufacturing procedure, a third set of control signals from the NFC reader device instructing the AFE component to output a fourth set of control signals to the power control sensor to close the power switch, wherein:
based on the fourth set of control signals from the AFE component, operating the power switch comprises closing the power switch to allow the current flow from the battery to at least the transcutaneous analyte sensor, the AFE component, and the one or more other electrical components.

18. The method of claim 17, further comprising performing, by the one or more other electrical components, one or more testing procedures associated with the analyte sensor system based on the third set of control signals received from the NFC reader device and the current flow from the battery.

19. The method of claim 15, further comprising:
detecting, by the one or more other electrical components, a failure of the analyte sensor system; and
outputting, by the one or more other electrical components based on the detected failure, a third set of control signals instructing AFE component to output a fourth set of control signals to the power control sensor to open the power switch; and
based on the fourth set of control signals, operating the power switch comprises opening the power switch to prevent the current flow from the battery to at least the transcutaneous analyte sensor, the AFE component, and the one or more other electrical components.

20. An analyte monitoring system, comprising:
an analyte sensor system configured to perform analyte measurements associated with a user of the analyte sensor system; and
a display device configured to receive the analyte measurements from the analyte sensor system, wherein:
the analyte sensor system includes:
a power switch;
a power control sensor;
an analog front end (AFE) component; and
one or more other electrical components;
the power control sensor is configured to detect whether a signal is applied to the power control sensor and, based on the detection, operate the power switch to control current flow from a battery of the analyte sensor system to at least a transcutaneous analyte sensor of the analyte sensor system, the AFE component, and the one or more other electrical components;
the power control sensor is configured to receive a first set of control signals from the AFE component for instructing the power control sensor to operate the power switch; and
the AFE is configured to receive a second set of control signals from at least one of a near field communication (NFC) reader device or the one or more other electrical components instructing the AFE to output the first set of control signals for instructing the power control sensor to operate the power switch.
